(19) United States
(12) Patent Application Publication
(10) Pub. No.: US 2023/0355194 A1
(43) Pub. Date: Nov. 9, 2023

(54) Medical Imaging Device And Methods

(71) Applicant: Mobius Imaging, LLC, Shirley, MA (US)

(72) Inventors: Eugene A. Gregerson, Bolton, MA (US); Russell Stanton, Lunenburg, MA (US)

(73) Assignee: Mobius Imaging, LLC, Shirley, MA (US)

(21) Appl. No.: 18/026,149

(22) PCT Filed: Sep. 15, 2021
(86) PCT No.: PCT/US2021/050403

§ 371 Date: Mar. 14, 2023

Related U.S. Application Data
(60) Provisional application No. 63/078,432, filed on Sep. 15, 2020.

(51) Int. Cl. A61B 6/00; A61B 6/06; A61B 6/04

(52) U.S. Cl. CPC A61B 6/4447; A61B 6/4476; A61B 6/54; A61B 6/06; A61B 6/4064; A61B 6/4458; A61B 6/0407; A61B 6/4266

(57) ABSTRACT

A medical imaging device including an imaging gantry supported in a medical facility and movable relative to a patient positioning device. The imaging device may further include a drive system coupled to the imaging gantry and operable to effect movement of the imaging gantry. The medical imaging device may further include a control system in communication with the drive system and configured to control movement of the imaging gantry.

### CROSS-REFERENCE TO RELATED APPLICATIONS

The subject patent application claims priority to and all the benefits of U.S. Provisional Patent Application No. 63/078,432, filed on Sep. 15, 2020, the entire contents of which are incorporated by reference herein.

### BACKGROUND

Hybrid operating rooms integrate traditional operating rooms with specialized medical imaging equipment, such as fixed-room C-arms, x-ray computed tomography (CT) devices, and magnetic resonance imaging (MM) systems. Hybrid operating rooms are intended to provide medical professionals with the flexibility to perform a wide variety of procedures, including open surgeries and minimally-invasive procedures (such as laparoscopy), often during the same patient visit. This may lead to improved patient outcomes and shorter recovery times.

Hybrid operating rooms are typically large and are expensive to build. The initial investment for implementing a hybrid operating room may be in excess of five million U.S. dollars, which includes the costs of the surgical and imaging equipment as well as the costs of constructing the hybrid operating theater.

### SUMMARY

The present teachings generally provide a medical imaging device. The medical imaging device may include an imaging gantry attached to a support surface, the imaging gantry comprising an O-shaped housing defining a bore and containing one or more image collection components configured to obtain imaging data from a patient located in the bore. The medical imaging device may include a support column that supports the imaging gantry relative to the support surface. The medical imaging device may further include a drive system comprising at least one drive motor that is operable to translate the imaging gantry along three perpendicular directions relative to the support surface and rotate the imaging gantry about three perpendicular axes relative to the support surface.

The present teachings may further provide a medical imaging device usable in a medical facility having a support surface. The medical imaging device may include an imaging gantry supported by the support surface, the imaging gantry comprising a gantry housing defining a bore and supporting one or more image collection components configured to obtain image data of a patient positioned in the bore. The medical imaging device may further include a support column coupled between the imaging gantry and the support surface. The medical imaging device may further include a drive system operably coupled to the imaging gantry and configured to effect translation of the imaging gantry relative to the support surface. The drive system may include a translation drive motor. The drive system may further include a control system comprising one or more controllers, the control system comprising a motion controller in communication with the translation drive motor and configured to send control signals to the translation drive motor to control movement of the imaging gantry relative to the support surface.

The present teachings may further provide a medical imaging device usable in a medical facility having a support surface. The medical imaging device may include an imaging gantry supported by the support surface, the imaging gantry comprising a gantry housing defining a bore and supporting one or more image collection components configured to obtain image data of a patient positioned in the bore. The medical imaging device may further include a robotic arm coupled to the imaging gantry for positioning an end effector usable during a medical procedure. The medical imaging device may further include a drive system operably coupled to the imaging gantry and comprising a drive motor configured to effect movement of the imaging gantry relative to the support surface. The medical imaging system may further include a control system comprising one or more controllers in communication with the drive motor and the robotic arm, the control system configured to send control signals to the translation drive motor to control movement of the imaging gantry relative to the support surface.

The present teachings may further provide a medical imaging device usable in a medical facility having a support surface. The medical imaging device may include an imaging gantry supported by the support surface, the imaging gantry comprising a gantry housing defining a bore and supporting one or more image collection components configured to obtain image data of a patient positioned in the bore. The medical imaging device may further include a drive system operably coupled to the imaging gantry and comprising a drive motor configured to effect movement of the imaging gantry relative to the support surface. The medical imaging device may further include a patient positioner movable between a first patient support position and a second patient support position to move the patient relative to the support surface. The medical imaging device may further include a control system including one or more controllers in communication with the drive motor and the patient positioner, the control system configured to send control signals to the patient positioner and the translation drive motor to control movement of the imaging gantry and the patient positioner relative to the support surface.

### BRIEF DESCRIPTION OF THE DRAWINGS

Other features and advantages of the present disclosure will be appreciated by reference to the following detailed description, taken in conjunction with the accompanying drawings.

FIG. 1A illustrates a medical imaging device and a patient positioner in a hybrid operating room according to one embodiment.

FIG. 1B illustrates an alternative view of the medical imaging device of FIG. 1A in a hybrid operating room.

FIG. 1C illustrates a medical imaging device with a robotic arm coupled to the medical imaging device and a patient positioner in a hybrid operating room according to another embodiment.

FIG. 1D illustrates an alternative view of the medical imaging device and the patient positioner of FIG. 1A in a hybrid operating room.

FIG. 1E illustrates an alternative view of the medical imaging device and the patient positioner of FIG. 1A in a hybrid operating room.

FIGS. 2A and 2B illustrate a 3D motion control input device for a medical imaging device.

FIGS. 3A-3C illustrate components of a gantry of a medical imaging device for performing x-ray fluoroscopy, cone-beam x-ray CT imaging and fan-beam x-ray CT imaging.

FIG. 4A illustrates an additional embodiment of a medical imaging device with a robotic arm coupled to the medical imaging device and a patient positioner in a hybrid operating room.

FIG. 4B illustrates the medical imaging device of FIG. 4A without the robotic arm in a hybrid operating room.

FIG. 4C illustrates an alternative view of the medical imaging device without the robotic arm of FIG. 4B.

FIG. 4D illustrates an alternative view of the medical imaging device without the robotic arm of FIG. 4B.

FIG. 5A illustrates a medical imaging device according to yet another embodiment.

FIG. 5B illustrates an alternative view of the medical imaging device of FIG. 5A in a hybrid operating room.

FIG. 6 schematically illustrates a computing device which may be used for performing various embodiments.

### DETAILED DESCRIPTION

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the invention or the claims.

Various embodiments include a medical imaging device and methods therefor. The medical imaging device may be a multi-modal medical imaging device. As used herein, a multi-modal imaging device is an imaging device that provides two or more medical imaging modalities in a single device, where the medical imaging modalities may include, for example, x-ray fluoroscopy, three-dimensional x-ray computed tomography (CT), magnetic resonance imaging (MM), positron emission tomography (PET), single-photon emission computed tomography (SPECT), and ultrasound imaging. The multi-modal medical imaging device according to various embodiments may be particularly suited for use in a hybrid operating room.

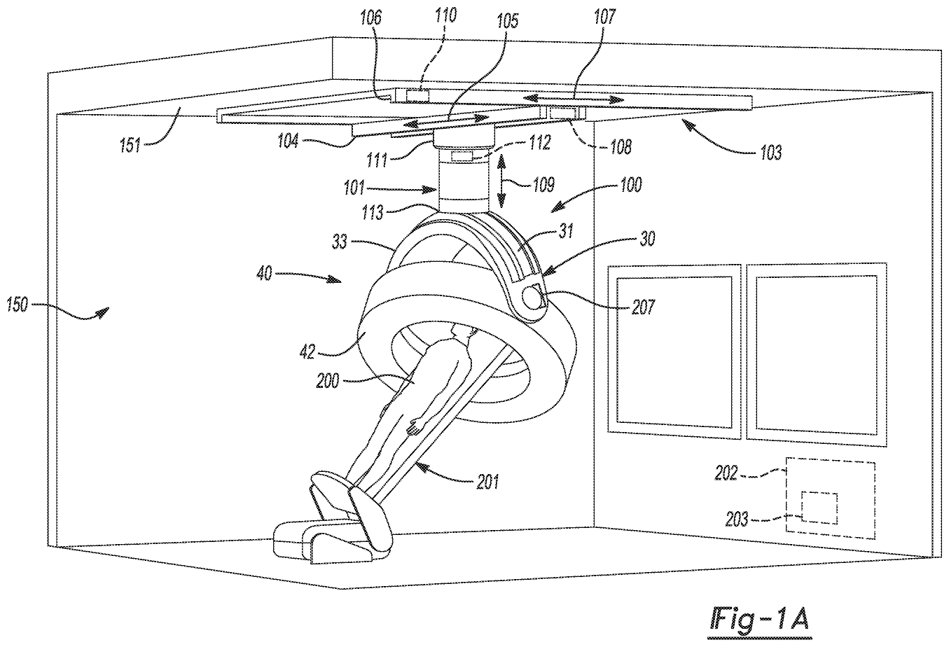

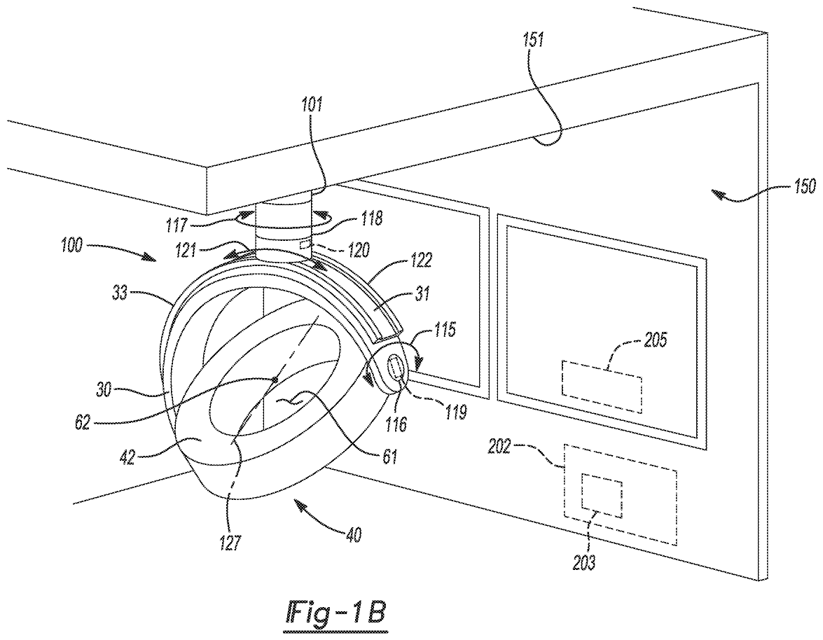

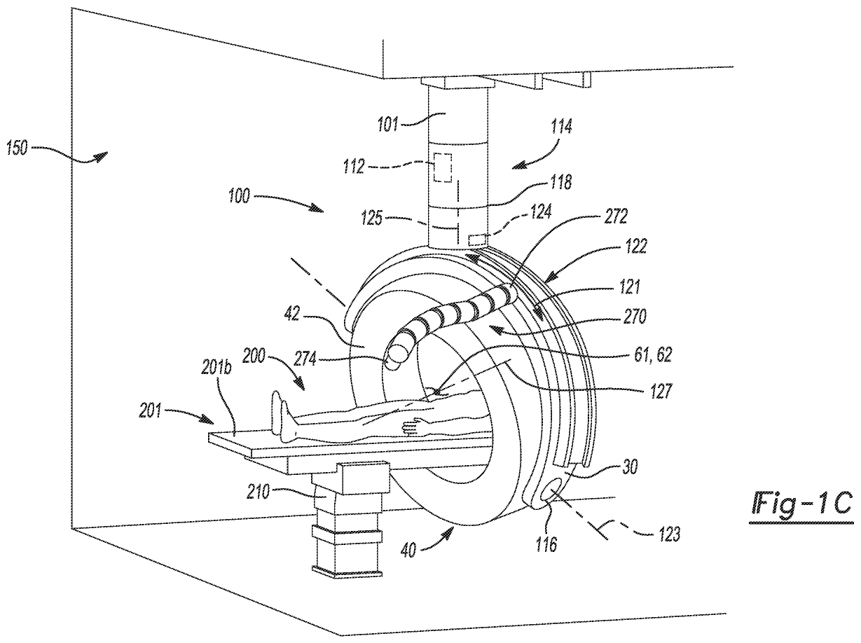

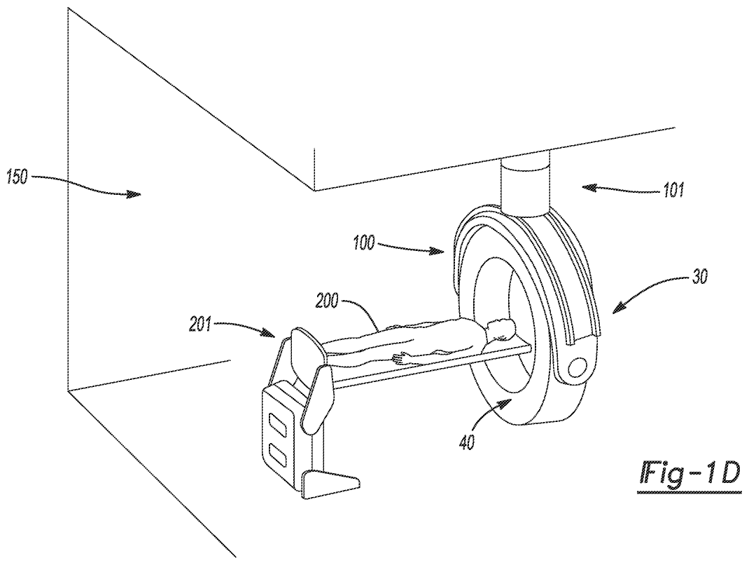

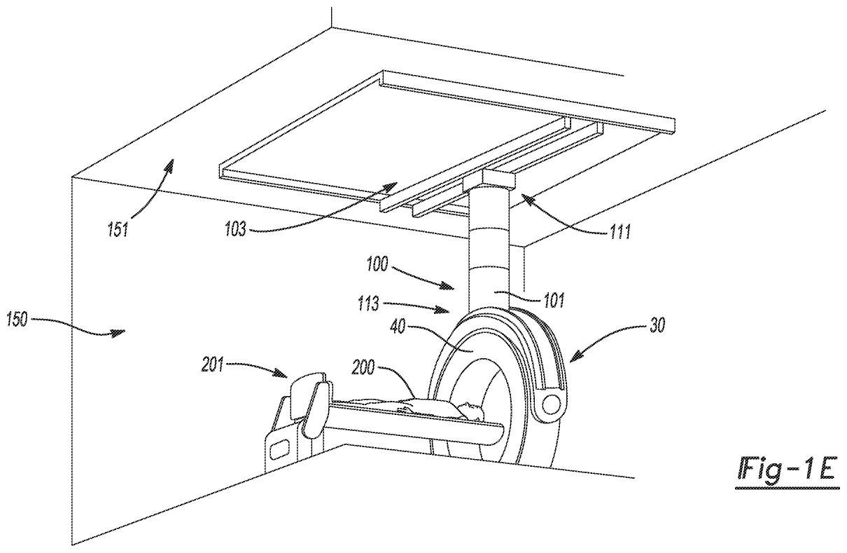

Referring to FIGS. 1A-1E, a medical imaging device 100 according to one embodiment of the invention is shown. The imaging device 100 is illustrated in a medical facility such as a hybrid operating room 150, although it will be understood that the imaging device 100 may be in other locations, such as in a radiology department, imaging center, emergency room, trauma center or other facility. The hybrid operating room 150 has a support surface 151. The imaging device 100 includes an imaging gantry 40 that is attached to the support surface 151. As shown in FIGS. 1A-1E, the support surface 151 is the ceiling of the room 150 in which the imaging device 100 is located. In other embodiments, the support surface 151 to which the imaging gantry 40 is attached may be a wall or floor surface of the room 150, a joist, or a building support beam.

The imaging gantry 40 includes image collection components, such as an x-ray source and detector array, a gamma-ray camera or magnetic resonance imaging components, that are housed within the gantry 40. The image collection components are configured to collect image data or imaging data, such as, for example x-ray fluoroscopy, computed tomography (CT), positron emission tomography (PET), single-photon emission computed tomography (SPECT) or magnetic resonance imaging (MRI) data, from an object located within a bore 61 of the gantry 40, in any manner known in the medical imaging field. As shown in FIGS. 1A-1E, the imaging gantry 40 may be a generally ring-shaped (e.g., shaped like an O) structure having a gantry housing 42 and internal housing or cavity that contains the image collection components. More specifically, the imaging gantry 40 may comprise the gantry housing 42, which may contain the image collection components. The gantry housing 42 defines the bore 61 in which the patient 200 may be positioned when obtaining image data. The bore 61 is generally circular in shape and has a center or isocenter 62.

In embodiments, the imaging device 100 may be used for acquiring x-ray images, and the imaging gantry 40 may include at least one x-ray source and at least one x-ray detector. The imaging gantry 40 may also include other components, such as a high-voltage generator, a heat exchanger, a power supply (e.g., battery system), and a computer. These components may be mounted on a rotating element (e.g., a rotor) that rotates within the imaging gantry 40 during an imaging scan. A rotor drive mechanism may drive the rotation of the rotor. The rotation of the rotor may enable the imaging components (e.g., the at least one x-ray source and at least one x-ray detector) to obtain image data of a patient 200 located within the bore 61 of the imaging gantry from a plurality of projection angles. A docking system may be used to couple the rotating and non-rotating portions of the imaging gantry for power and data communication. In other embodiments, a cable system or slip-ring may be used to provide power to the rotating portion of the imaging gantry. Data may be communicated between the rotating and non-rotating portions via a wired or wireless communication link.

In the imaging device 100 of FIGS. 1A-1E, the imaging gantry 40 is movable with respect to the support surface 151. In embodiments, the imaging device 100 may provide the imaging gantry 40 with six degrees-of-freedom, including translation along three perpendicular axes and rotation about three perpendicular axes. The imaging device 100 may provide similar imaging flexibility as a fixed-room C-arm while also enabling diagnostic-quality x-ray CT scans along multiple axes (e.g., horizontal, oblique, and in some cases vertical scans). A multi-modal medical imaging device as described and illustrated herein may provide a reduced footprint and reduce costs for a hybrid operating room.

In order to effect movement of the imaging gantry 40, the imaging device 100 may comprise a drive system operably coupled to the imaging gantry 40. The drive system may be configured to effect translation of the imaging gantry 40 in the direction of arrows 105, 107, and 109. The drive system may be further configured to effect rotation of the imaging gantry 40 relative to the support surface 151 in the direction of arrows 115, 117, 121. The drive system may comprise one or more drive motors, such as translation drive motors and rotation drive motors, discussed below, which provide motive power to move the imaging gantry 40.

As shown in FIGS. 1A-1E, the imaging gantry 40 is suspended from the support surface 151 by a support column 101 having a base end 111 and a distal end 113. In the embodiment illustrated, the distal end 113 of the support column 101 is coupled to a gimbal 30 that includes a pair of arms 31, 33 extending away from the support column 101. Each of the arms 31, 33 of the gimbal 30 is rotatably coupled to the imaging gantry 40 on a common axis of rotation, as will be discussed in further detail below.

The base end 111 of the support column 101 is attached to a linear motion system 103 that is mounted to the support surface 151. The linear motion system 103 is coupled between the support surface 151 and the support column 101 and configured to constrain movement of the imaging gantry 40 and the support column 101 in two degrees of freedom relative to the support surface 151. In the embodiment shown in FIGS. 1A-1E, the linear motion system 103 is a two-axis linear stage system that is mounted to the ceiling of the room 150. The linear motion system 103 enables the imaging gantry 40, gimbal 30 and support column 101 to translate along two perpendicular directions. As shown in FIG. 1A, the movement of the support column 101 on a first linear bearing assembly 104 translates the imaging gantry 40 in the direction of arrow 105. The movement of the first linear bearing assembly 104 and support column 101 on a second linear bearing assembly 106 translates the imaging gantry 40 in the direction of arrow 107.

A first translation drive motor 108 may drive the translation of the imaging gantry 40 along the direction of arrow 105 and a second translation drive motor 110 may drive the translation of the imaging gantry 40 along the direction of arrow 107. Said differently, the first translation drive motor 108 may be coupled to the first linear bearing assembly 104 and the second translation drive motor 110 may be coupled to the second linear bearing assembly 106.

The support column 101 may also include a second linear motion system that enables the imaging gantry 40 to translate along the direction of arrow 109. The second linear motion system may be coupled between the support surface 151 and the support column 101 and configured to constrain movement of the imaging gantry 40 relative to the support surface 151 in a third degree of freedom. As shown in FIG. 1C, the linear motion system may include a telescoping portion 114 of the support column 101 that may be utilized to adjust the displacement of the imaging gantry 40 towards or away from the support surface 151. A third translation drive motor 112 may drive the translation of the imaging gantry along the direction of arrow 109.

FIG. 1B illustrates the rotational degrees-of-freedom of the imaging gantry 40 of the imaging device 100. The imaging gantry 40 may be attached to the arms 31, 33 of the gimbal 30 by a pair of rotary bearings 116 that enable the imaging gantry 40 to rotate (i.e., tilt) with respect to the gimbal 30 about a pitch axis 123, generally in the directions indicated by arrow 115. An additional rotary bearing 118 may enable the gimbal 30 and imaging gantry 40 to rotate with respect to the support surface 151 about a yaw axis 125, generally in the directions indicated by arrow 117. Rotary bearing 118 may be located in the support column 101 as shown in FIG. 1B. Alternately, the rotary bearing 118 may be located at the interface between the gimbal 30 and the support column 101, or at the interface between the support column 101 and the linear motion system 103. A first rotation drive motor 119 may drive the rotation of the imaging gantry 40 along the direction of arrow 115 and a second rotation drive motor 120 may drive the rotation of the imaging gantry 40 along the direction of arrow 117.

As shown in FIGS. 1B and 1C, the imaging device 100 also includes a curved bearing assembly 122 coupled between the distal end 113 of the support column 101 and the gimbal 30 that enables the gimbal 30 and imaging gantry 40 to rotate with respect to the support surface about a roll axis 127, generally in the directions indicated by arrow 121. The bearing assembly 122 may extend in an arc over an outer surface of the gimbal 30 and may follow a curved path that is centered on the isocenter 62 of the imaging gantry 40.

The rotation of the gimbal 30 and imaging gantry 40 on the curved bearing assembly 122 may be within an imaginary plane that contains or is defined by the pitch axis 123 about which the imaging gantry 40 tilts on rotary bearings 116 and the yaw axis 125 about which the gimbal 30 and imaging gantry 40 rotate on rotary bearing 118, as shown in FIG. 1C. In embodiments, the gimbal 30 and imaging gantry 40 may rotate between a base position in which the pitch axis 123 is perpendicular to the yaw axis 125 and an offset position in which the pitch axis 123 is not perpendicular to the yaw axis 125. The gimbal 30 and imaging gantry 40 may rotate over an angular range between the base position and a desired offset position, such as up to 30° from the base position or more, including up to 45°, 60°, 90°, or 150° from the base position. The gimbal 30 and imaging gantry 40 may be rotatable away from the base position in a single direction or bi-directionally (e.g., ±45°). A third rotation drive motor 124 may drive the rotation of the gimbal 30 and imaging gantry 40 about the roll axis 127 and generally along the direction of arrow 121.

The rotation of the imaging gantry 40 may be isocentric in all degrees of rotational freedom, meaning that as the imaging gantry 40 rotates along the direction of any of arrows 115, 117 and 121, the axes 123, 125, 127 of rotation of the imaging gantry 40 all intersect at a point (i.e. the isocenter 62) in the center of the bore 61, which may remain stationary relative to the patient 200 as the imaging gantry 40 rotates along the direction of any of arrows 115, 117 and 121 (i.e. about any of the axes 123, 125, 127). In embodiments, the isocenter 62 may also be intersected by the central ray of an imaging radiation beam (e.g., an x-ray beam in the case of an x-ray imaging device) as the imaging gantry 40 rotates along the direction of any of arrows 115, 117 and 121.

The medical imaging device 100 may further comprise a control system 202, which may control motion, position, movement, or operation of the imaging gantry 40. As will be discussed in further detail below, the control system 202 may further control motion, position, movement, or operation of a robotic arm 270 and a patient positioner 201. The control system 202 may comprise one or more discrete controllers that are in communication with each other or are integrated into a single controller. One exemplary controller is a motion controller 203, which may be in communication with the drive system. Additional controllers such as a patient position controller and an arm controller are contemplated.

A motion controller 203 and control system 202, schematically illustrated in FIG. 1B, may be operatively coupled to the imaging device 100, including the image collection components in the imaging gantry 40, and may control the operation of the imaging device 100. The motion controller 203 may be implemented using one or more processing devices (e.g., computers) configured with software and/or firmware that is operable to control various functions of the imaging device 100. The motion controller 203 may send control signals to each of the first translation drive motor 108, the second translation drive motor 110, the third translation drive motor 112, the first rotation drive motor 119, the second rotation drive motor 120, and the third rotation drive motor 124 to control the various motions of the imaging gantry 40 as described above. The control system 202 and motion controller 203 may further support coordinated motion of the drive motors to enable the imaging gantry 40 to perform complex movements, such as performing an imaging scan along a particular trajectory. The motion controller 203 may also receive feedback from the imaging device 100, such as encoder data, that indicates the current state of the imaging device 100. Also shown schematically in FIG. 1B is a control console 205, which may be coupled to the motion controller 203 and may enable a user to control the operation of the imaging device 100. The control console 205 may be a workstation, which may be located outside of the room 150 (e.g., behind suitable lead shielding), as shown in FIG. 1B. Alternately or in addition, a control console for controlling at least a portion of the functionality of the imaging device 100 may be located on the imaging device 100 itself, on a mobile cart, or on a handheld device, such as a pendant controller.

Also illustrated in FIGS. 1A, 1D-1E, 4A-4D, and 5B is a patient positioner 201 that may support the patient 200 while the imaging device 100 obtains images of the patient 200. FIG. 1A illustrates a patient positioner 201 that may be used for obtaining images of a patient 200 in a first patient support position (e.g., a weight-bearing standing position as shown in FIG. 1A) as well as in a second patient support position (e.g., a lying position as shown in FIGS. 1D-1E). An example of a patient positioner 201 such as shown in FIGS. 1A, 1D-1E, 4A-4D, and 5B is described in U.S. Pat. No. 10,980,692 B2, the entire contents of which are incorporated by reference herein.

As can be seen in perspective views 1C and 4A, some configurations of the system may include at least one robotic arm 270 that is movable between a first arm pose and a second arm pose with respect to the patient 200. It will be understood that in other examples, the system may include two or more robotic arms. The movement of the robotic arm 270 may be controlled by the control system 202, which may include one or more arm controllers (not shown), which may be coupled to the robotic arm, the imaging device, the table, or a combination thereof via a wired or wireless link. Exemplary arm controllers may take the form of a computer, comprising a memory and processor for executing software instructions stored in or on the memory.

The robotic arm 270 may comprise a multi joint arm that includes a plurality of linkages connected by joints having actuator(s) and optional encoder(s) to enable the linkages to bend, rotate and/or translate relative to one another in response to control signals from the control system 202. A first end 272 of the robotic arm 270 may be fixed to a mounting structure coupled to the gantry housing 42 and a second end 274 of the robotic arm 270 may be freely movable with respect to the first end 272. An end effector (not shown) may be attached to the second end 274 of the robotic arm 270 such that the robotic arm 270 is able to position the end effector during a medical procedure. In some embodiments, the end effector may be an invasive surgical tool, such as a needle, a cannula, a cutting or gripping instrument, an endoscope, etc., that may be inserted into the body of the patient. In other embodiments the end effector of the robotic arm 270 may be a hollow tube or cannula that may receive an invasive surgical tool, including without limitation a needle, a cannula, a tool for gripping or cutting, an electrode, an implant, a radiation source, a drug, and an endoscope. The invasive surgical tool may be inserted into the patient's body through the hollow tube or cannula by a surgeon. An end effector comprising a hollow tube or cannula may be made of a radiolucent material, such as a carbon-fiber or thermoplastic material.

The patient 200, which may be a human or animal patient, may be located on a suitable patient positioner 201, which may be a surgical table 201b as shown in FIG. 1C. The patient positioner 201 in this embodiment is raised off the ground by a support column 210. During a surgical procedure, the robotic arm 270 may be located partially or completely within the sterile surgical field, and thus may be covered by a surgical drape or other sterile barrier (not shown for clarity). In embodiments, the end effector (e.g., a hollow tube or cannula) may be a sterilized component that may be attached (e.g., snapped into) the second end 274 of the robotic arm 270 over the drape. The end effector may be a sterile, single-use (i.e., disposable) component that may be removed and discarded after use.

The control system 202 may control the at least one robotic arm 270 to move the end effector of the robotic arm 270 to a pre-determined position and orientation with respect to the patient 200. Said differently, the robotic arm 270 is movable between at least a first arm pose and a second arm pose in order to optimally position the end effector for use during a medical procedure. The pre-determined position and orientation may be based on imaging data obtained by the imaging device 100. For example, the imaging data may be used to determine a unique vector in three-dimensional space corresponding to a desired insertion trajectory for a surgical tool, such as described in U.S. Pat. No. 10,959,783 B2, the entire contents of which are incorporated by reference herein.

In some examples, a motion tracking apparatus such as described above may be configured to track the at least one robotic arm 270 to ensure that the end effector maintains the pre-determined position and orientation with respect to the patient 200. If an end effector moves from the pre-determined position and orientation (e.g., due to the robotic arm 270 being accidentally bumped), the motion tracking apparatus may detect this movement and alert the surgeon or other clinician. Alternately or in addition, the motion tracking apparatus may send a message to the control system 202 of the at least one robotic arm 270 indicating a detected deviation from the pre-determined position and orientation of the end effector. The control system 202 may then move the robotic arm 270 to compensate for the detected deviation. In some examples, the motion tracking apparatus may also track the patient 200 (e.g., where a plurality of markers are placed on the patient) to determine whether the patient 200 has moved relative to the end effector. The motion tracking apparatus may notify the surgeon when the patient 200 moves by more than a pre-determined amount. In some embodiments, the motion tracking apparatus may send message(s) to the control system 202 of the robotic arm(s) 270 regarding detected movements of the patient 200. The control system 202 may move the robotic arm(s) 270 to compensate for any such movement (e.g., to maintain the end effector in the same position and orientation with respect to the selected entrance point on the patient's body).

In embodiments, the control system 202 may control the movement of the robotic arm 270 such that the arm 270 does not collide with either the imaging gantry 40, the patient positioner 201, or the patient 200 during the movement of the arm 270. For example, as the imaging gantry 40 and the robotic arm 270 move from one position another, at least a portion of the robotic arm 270 including the end effectors may be located inside the bore 61 of the imaging gantry 40. The control system 202 may control the movement of the robotic arm 270 so that as the imaging gantry 40 advances towards the patient, none of the joints of the robotic arm 270 collide with the side wall or inner diameter of the ring or with the patient 200. The control system 202 may control the movement(s) of the arm(s) 270 in accordance with a motion planning algorithm and collision model that utilizes inverse kinematics to determine the joint parameters of the robotic arm 270 that maintain the position and orientation of the end effector while avoiding collisions with the imaging gantry 40 and the patient 200.

In some examples, the control system 202 may determine the position of the robotic arm 270 in relation to the imaging gantry 40 based on position data received from the motion controller 203 (e.g., indicating the translation and/or tilt position of the gantry 40 with respect to the base support column 101). Alternately or in addition, the control system 202 may utilize position information received from the motion tracking apparatus. As discussed above, the motion tracking system may be used to construct a three-dimensional model (e.g., a CAD model) of the various objects being tracked by the motion tracking apparatus. The sharing of data between the robotic system, the imaging device, the patient positioner 201, and the motion tracking apparatus may enable these systems and the control system 202 to operate in a common coordinate system.

In some cases, the control system 202 may determine that it is not possible to move a robotic arm 270 without either changing the position or orientation of the end effector with respect to the patient 200, or without some part of the robotic arm 270 colliding with the imaging gantry 40 or the patient 200. For example, a translation of the imaging gantry 40 may result in the arm 270 being extended beyond its maximum length. In other cases, the control system 202 may determine that no set of joint movements are possible to avoid collisions while maintaining the end effector in a fixed position and orientation. In such a case, the control system 202 may issue an alert (for example via the control counsel 205), which may be perceived by the surgeon or other clinician, and may also send a signal to the motion controller 203 to stop the motion of the imaging gantry 40.

In some implementations a support member (not shown) may extend from the gimbal 30 (e.g., from one of the arms 31, 33 of the gimbal 30) and at least one robotic arm 270 may be mounted to the support member. In some examples, the support member may extend at least partially around an outer circumference of the gantry 40. For example, the support member may comprise a curved rail that extends around the outer circumference of the gantry 40. In this example, the support member forms a semicircular arc that extends between the ends of the respective arms 31 and 33 of the gimbal 30. The semicircular support member may be concentric with the outer circumference of the imaging gantry 40.

A bracket mechanism may be located on the support member and may include a mounting surface for mounting the first end 272 of the robotic arm 270 to the bracket mechanism. The mounting surface may project from the side of the support member and may be upwardly angled. This may provide additional clearance for the “tilt” motion of the gantry 40 relative to the gimbal 30.

The bracket mechanism and the robotic arm 270 attached thereto may be moved to different positions along the length of support member (e.g., any arbitrary position between the ends of the arms 31, 33 of the gimbal 30) and may be fixed in place at a particular desired position along the length of the support member. In some embodiments, the bracket mechanism may be moved manually (e.g., positioned by an operator at a particular location along the length of the support member and then clamped or otherwise fastened in place). Alternately, the bracket mechanism may be automatically driven to different positions using a suitable drive mechanism (e.g., a motorized belt drive, friction wheel, gear tooth assembly, cable-pulley system, etc.). The drive mechanism may be located on the bracket mechanism, the support member and/or the gimbal 30, for example. An encoder mechanism may be utilized to indicate the position of the bracket mechanism and the first end 272 of the robotic arm 270 on the support member.

It will be understood that various types of patient positioners may be used with a multi-modal imaging device 100 according to the present disclosure, including, for example, operating and/or radiology table systems. FIG. 1C illustrates an alternative patient positioner 201 that includes a surgical tabletop 201b fixed to a support column 210. The imaging gantry 40 may be moved over the cantilevered end(s) of the tabletop 201b to obtain images of the patient 200 from a desired angle. The imaging gantry 40 may be moved away from the patient 200 when not in use so as not to obstruct a surgical or other medical procedure.

In embodiments, the control system 202 of the imaging device 100 may be operatively coupled to the patient positioner 201 or a patient position controller so that motion of the imaging gantry 40 and patient positioner 201 may be coordinated. The motion controller 203 may receive feedback from the patient positioner 201 that indicates the current position of the patient positioner 201 such as position data and optionally motion or movement data that includes any planned or current movement(s) of the patient positioner 201. In some embodiments, the motion controller 203 may implement a collision model to prevent the imaging device 100 from colliding with the patient positioner 201 or the patient 200. The collision model may enforce a set of rules that govern the permissible positions and motions of the imaging system 100 to avoid any portion of the imaging system 100 contacting the patient positioner 201 or patient 200. This may include, for example, controlling the position of the imaging gantry 40 so that the gimbal 30 and imaging gantry 40, including the surface of the gantry 40 surrounding the bore 61, maintain a minimum distance from the patient positioner 201. In some embodiments, the imaging device 100 may be controlled automatically to move in response to a movement of the patient positioner 201 in order to maintain a pre-determined spacing between the patient positioner 201 and the imaging gantry 40. The collision model may also include one or more bounding volumes around the patient positioner 201 that account for the patient 200 or other objects that are located on, or are attached to, the patient positioner 201. The motion controller 203 may further control the imaging device 100 to prevent any portion of the imaging device 100 from entering the boundary volume(s).

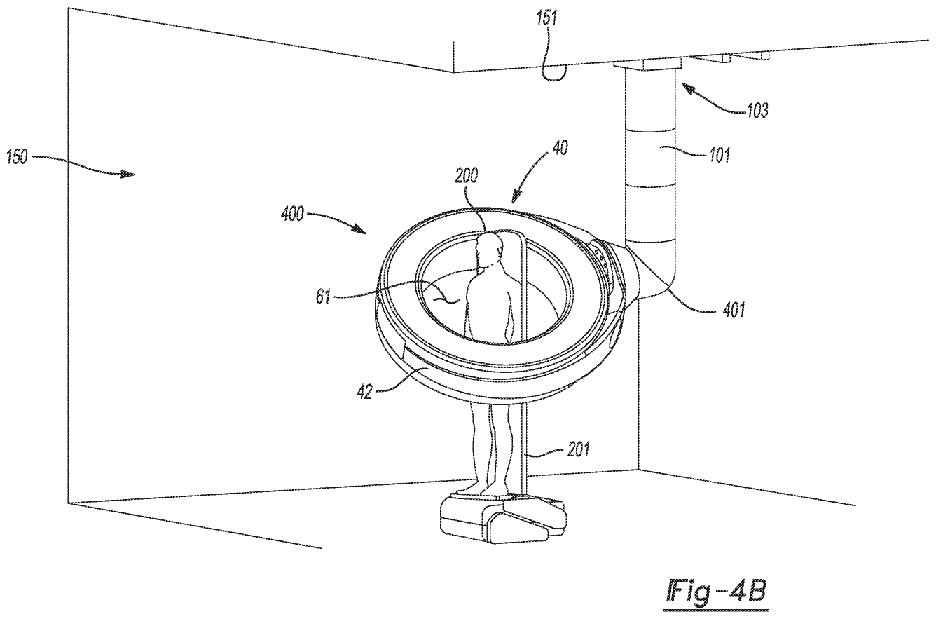

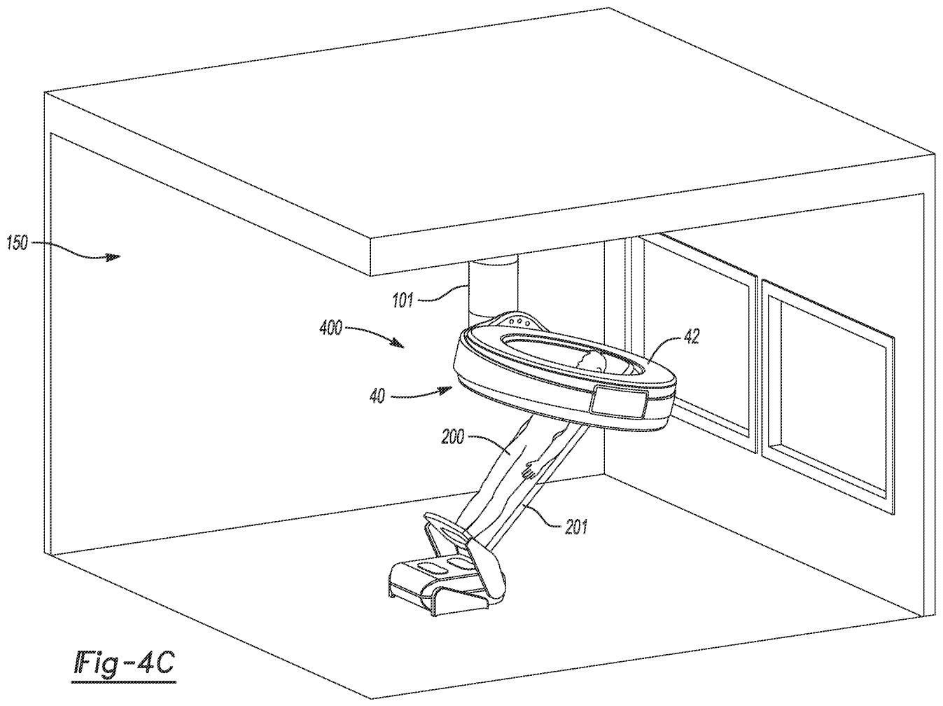

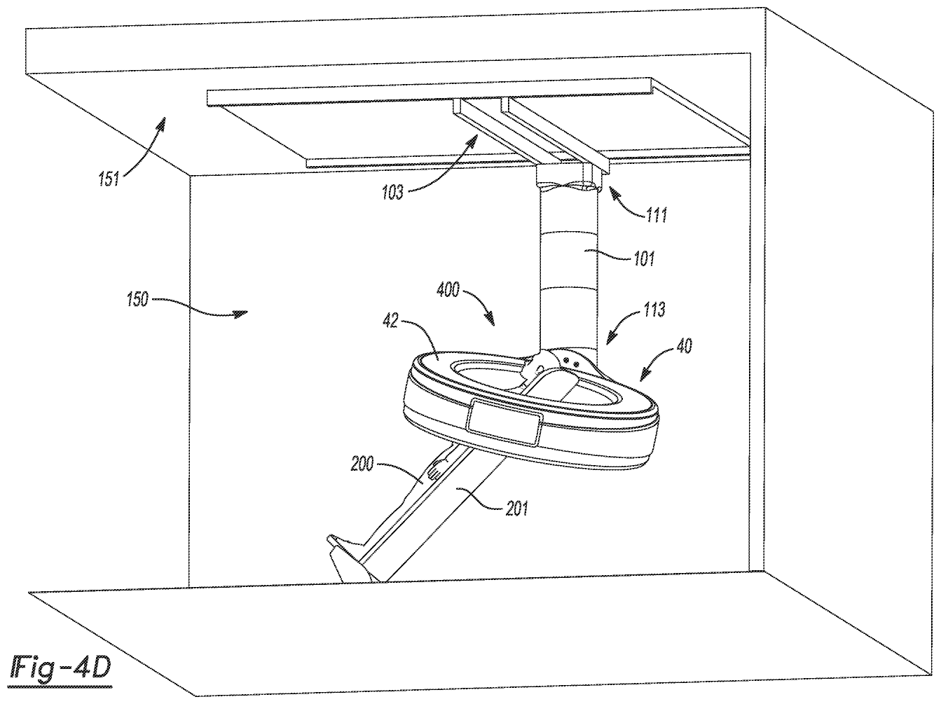

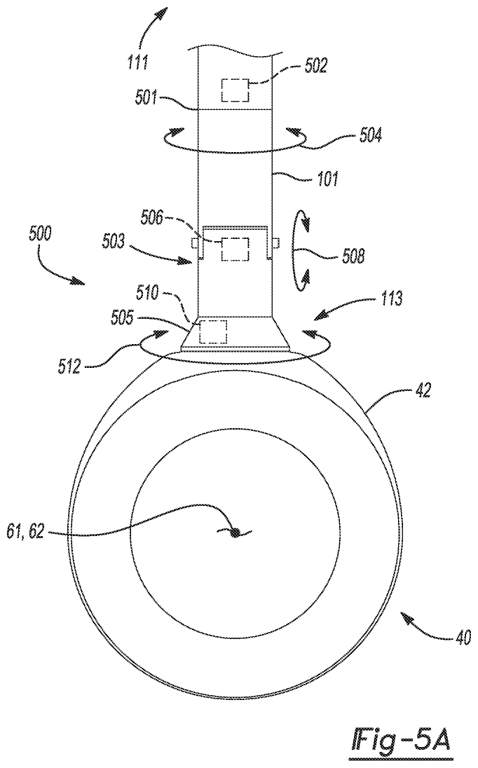

Similarly, the control system 202 including the motion controller 203, a patient position controller, and a robotic arm controller may in communication with one another in order to exchange position data so as to permit coordinated motion of the imaging gantry 40, the patient positioner 201, and the robotic arm 270. Referring to FIG. 4A, for example, the patient positioner 201 is shown in a second patient support position (e.g., a lying position) with the robotic arm 270 extended over the patient's head. If it is desired to obtain image data of the patient's upper torso with the patient in a second patient support position (e.g., a weight bearing position as shown in FIG. 1A) using the imaging device 100 as shown, it may necessitate moving the imaging gantry 40, the patient positioner 201, and the robotic arm 270. More specifically, it these movements may be required in order to obtain image data of the patient 200 during particular conditions, to maintain a particular angle of the end effector relative to the patient 200, and to avoid any collisions between any of the imaging gantry 40, the patient positioner 201, and the robotic arm 270 and obstructions in the room 150 such as walls or other equipment. The control system 202 may receive position data and motion data including speed or acceleration from the motion controller 203, the patient position controller, and the robotic arm controller in order to calculate an optimal path for each of the imaging gantry 40, the patient positioner 201 and the robotic arm 270. With these data the control system 202 may transmit control signals to at least one of the motion controller 203, the patient position controller, and the robotic arm controller to effect simultaneous operation of the imaging gantry 40, the patient positioner 201 and the robotic arm 270.

In embodiments, one or more proximity sensors may be operatively coupled to the motion controller 203 to prevent the imaging device 100 from colliding with other objects. The one or more proximity sensors may be optical (e.g., IR), ultrasonic, impedance, or capacitive-based sensors, for example, and may be located on the imaging device 100 and/or on other objects within the room 150. Feedback from the one or more proximity sensors indicating that a collision between the imaging device 100 and another object is imminent may cause the motion controller 203 to stop movement of the imaging device 100 and optionally move the imaging device 100 away from the object.

In some embodiments, at least one force sensor may be operatively coupled to the motion controller 203. The at least one force sensor may be located on the imaging device 100 and/or other objects within the room 150 (such as the patient positioner 201). The at least one force sensor may be a six-axis force-torque sensor that measures forces in three coordinate axes as well as three rotational axes. The motion controller 203 may receive feedback from the at least one force sensor to determine the forces applied to the imaging device 100. The motion controller 203 may utilize the feedback from the at least one force sensor to control the imaging device 100. For example, when the at least one force sensor detects an unanticipated force on the imaging device 100 while the device is moving, this may indicate that the imaging device 100 has collided with another object. In response to detecting such an unanticipated force, the motion controller 203 may stop the movement of the imaging device 100 and may optionally control the imaging device 100 to move in a direction opposite to the direction of the detected force until the force is sufficiently reduced or no longer detected.

In some embodiments, at least one force sensor as described above may be utilized to allow a user to manually move the imaging device 100. For example, the motion controller 203 may enter a hand guidance mode of operation of the imaging device 100, which may be in response to a user input command (e.g., a button-push, voice command, etc.). While operating in hand guidance mode, the motion controller 203 may receive feedback indicating the force and/or torque detected by the at least one force sensor, and in response control the drive motor(s) of the imaging device 100 to perform a corresponding movement of the imaging device 100 (e.g., a translation and/or rotation of the imaging gantry 40) in the direction of the applied force/torque. This process may occur repeatedly so that the user may move the imaging gantry 40 to a desired location and orientation. The motion controller 203 may control the imaging device 100 to move with a velocity and/or acceleration that is related to the magnitude of the force/torque detected by the at least one force sensor, and may be further configured to compensate for forces due to gravity such that the user may experience substantially the same resistance from the imaging device 100 when moving the device in any direction.

The imaging device 100 may include a handle 207 or other structure that the user may easily grip when moving the imagine device 100 in a hand guidance mode, as shown in FIG. 1A. The handle 207 may include a button or other sensor, which may be activated by a user to enter the hand guidance mode of operation. The motion controller 203 may only allow operation in hand guidance mode while the button/sensor is activated and may further restrict movements of the imaging device 100 to avoid collisions in accordance with a collision model and/or feedback from proximity sensor(s), as described above.

Alternatively or in addition, a user may control movement of the imaging device 100 using a 3D motion control input device 209, which may be a 3D mouse, that is operably coupled to the motion controller 203, for example. Examples of 3D mouse devices include the SpaceMouse® line of products from 3Dconnexion, Munich, DE. A 3D motion control input device 209 may include a moveable element, such as a knob, ball, joystick, or cap, on a base. Manipulation of the moveable element with respect to the base, including translation and/or rotational movements of the element, generates corresponding control signals that may be used to control another device.

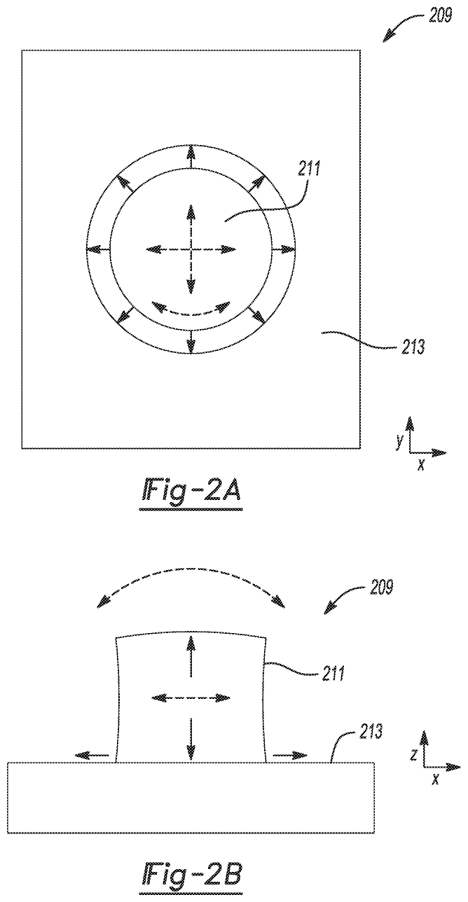

An example of a 3D motion control input device 209 for use with an imaging device 100 as described above is illustrated in FIGS. 2A and 2B. FIGS. 2A and 2B are top and side views, respectively, of the 3D motion control input device 209 according to one embodiment. The 3D motion control input device 209 includes a moveable element 211 coupled to a base 213. In this embodiment, the moveable element 211 is shown as a generally cylindrically-shaped knob-element that extends above a top surface of the base 213. A user may manipulate the knob 211 with respect to the base 213 in the X-Y plane as indicated by the coordinate arrows in FIG. 2A. In addition, the user may move the knob 211 towards and away from the base 213 in the direction of the Z-axis as shown by the coordinate arrows in FIG. 2B. The user may also rotate the knob 211 with respect the base 213 about the Z-axis and may tilt the knob 211 with respect to the base 213 about the X- and Y-axes as indicated by the dashed arrows in FIGS. 2A and 2B. The knob 211 may be coupled to the base 213 via spring or rubber-elastic elements that may bias the knob 211 to return to the “home” position as shown in FIGS. 2A and 2B when no pressure is being applied to the knob 211.

The 3D motion control input device 209 may include electronic circuitry that converts the various movements of the knob 211 as described above into electronic control signals that may be transmitted to the motion controller 203 and used to control the movements of the imaging device 100. In one non-limiting example, movement of the knob 211 along the X-axis may control the imaging device 100 to translate the imaging gantry 40 in the direction of arrow 105 (see FIG. 1A), movement of the knob 211 along the Y-axis may control the imaging device 100 to translate the imaging gantry 40 in the direction of arrow 107, and movement of the knob 211 towards or away from the base 213 in the direction of the Z-axis may control the imaging device 100 to translate the imaging gantry 40 in the direction of arrow 109. In addition, rotation of the knob 211 about the Z-axis may control imaging device 100 to rotate the gimbal 30 and imaging gantry 40 in the direction of arrow 117 (see FIG. 1B), tilting the knob 211 about the X-axis may control the imaging device 100 to tilt the imaging gantry 40 in the direction of arrow 115, and tilting the knob 211 about the Y-axis may control the imaging device 100 to rotate the gimbal 30 and imaging gantry 40 in the direction of arrow 117.

A 3D motion control input device 209 as disclosed herein may be included in a control console 205 for the imaging device 100, as shown in FIG. 1B. The control console 205 with 3D motion control input device 209 may be part of a workstation or on a mobile cart, for example. In some embodiments, a 3D motion control input device 209 such as shown in FIGS. 2A and 2B may be located on the imaging device 100, such as on the imaging gantry 40 or gimbal 30. In embodiments, the input device 209 may be included on a handheld control device (e.g., a pendant controller) that may be connected to the motion controller 203 via a wired or wireless link.

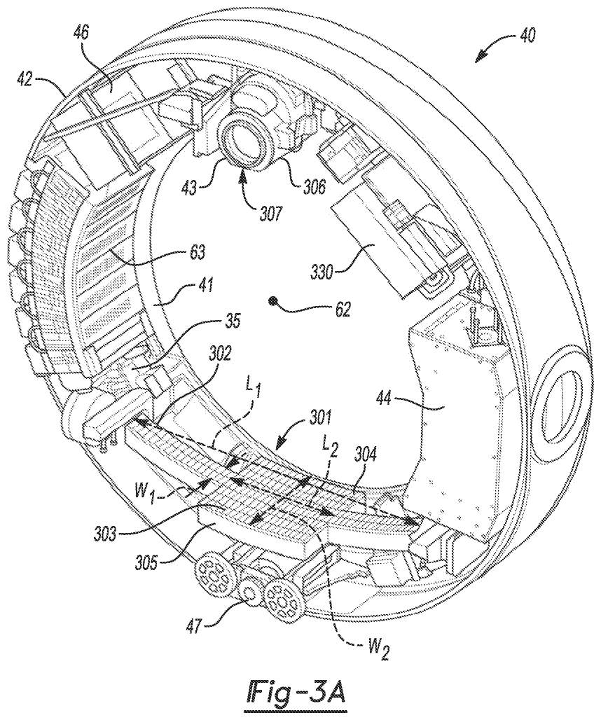

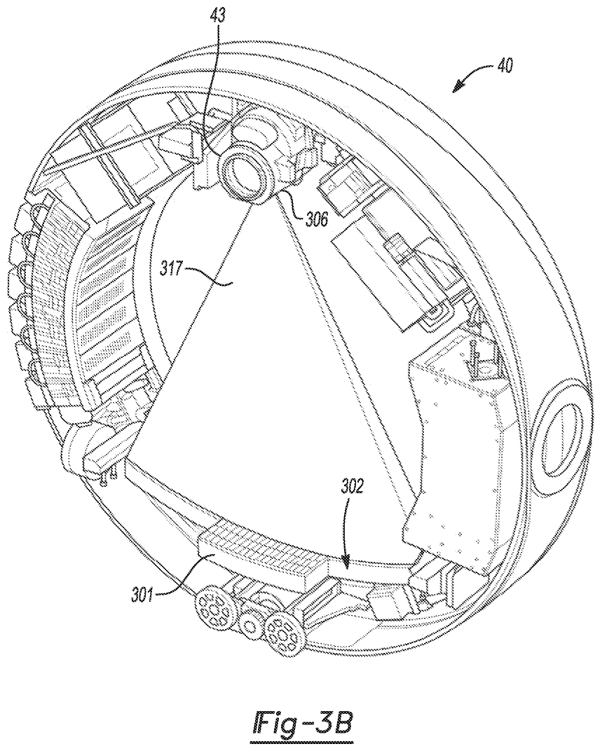

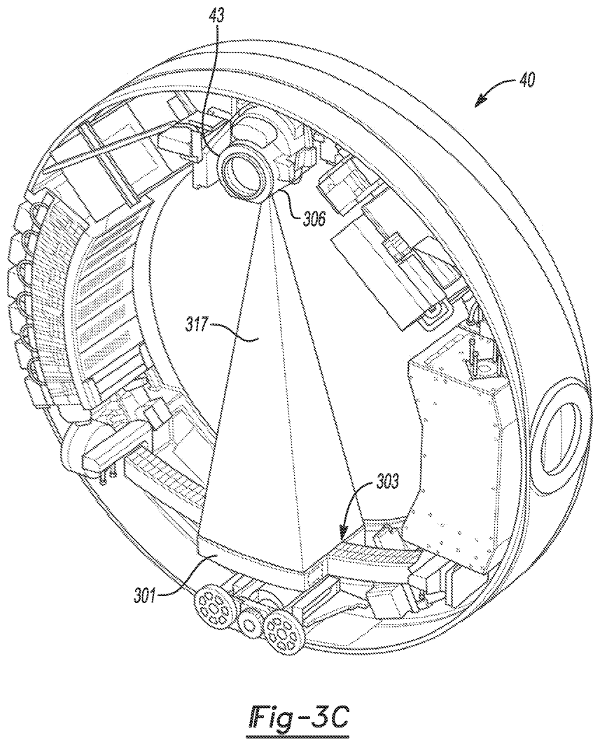

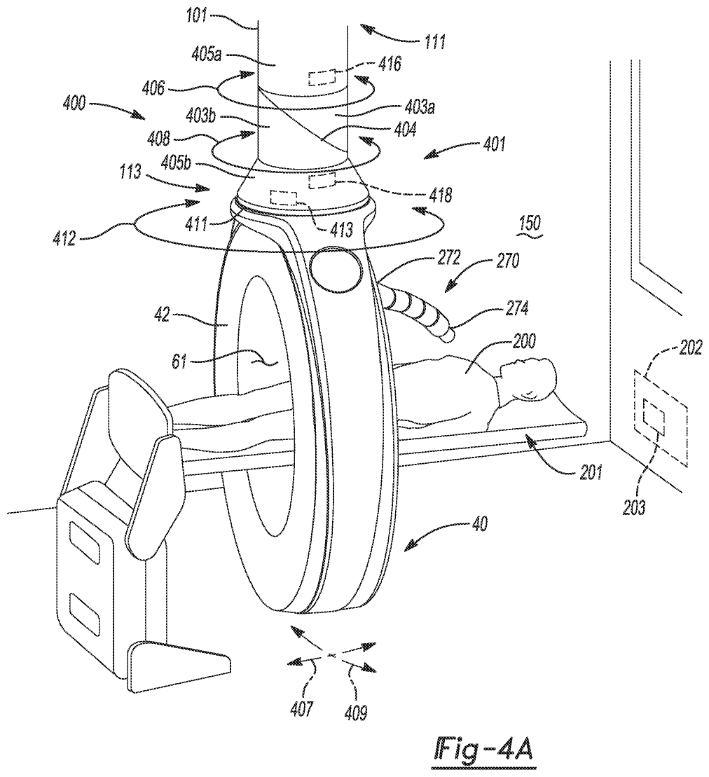

FIGS. 3A-3C are cutaway views of an imaging gantry 40 of a medical imaging device 100 according to one embodiment. In this embodiment, the imaging device 100 may perform two-dimensional x-ray fluoroscopic imaging as well as three-dimensional fan-beam x-ray CT imaging using a single x-ray source 43 and a single x-ray detector system 301 located in the imaging gantry 40. The detector system 301 in this embodiment may include a contiguous detector area that comprises an elongated first portion 302 for performing fan-beam CT imaging (e.g., axial and/or helical CT scans) and a panel-shaped second portion 303 for performing 2D fluoroscopic imaging and/or 3D cone beam CT imaging. An example of such a detector system 301 is shown and described in U.S. Patent Application Publication No. 2019/0282185 A1, the entire contents of which are incorporated herein.

The imaging gantry 40 illustrated in FIGS. 3A-3C includes a rotor 41 that is mounted to and rotates within the gantry housing 42 of the gantry 40. A plurality of components, including an x-ray source 43, a high-voltage generator 44, a heat exchanger 330, an x-ray detector system 301, a power supply 63 (e.g., battery system), a computer 46, a rotor drive mechanism 47, and a docking system 35, may be mounted to the rotor 41.

The detector system 301 in this embodiment includes a detector area having an elongated first portion 302 and a panel-shaped second portion 303, as noted above. The first portion 302 and the second portion 303 may be overlapping, such that a portion of the detector area is shared by both the first portion 302 and the second portion 303. The first portion 302 may have a length dimension L₁ that is greater than a length dimension L₂ of the second portion 303. For example, the first portion 302 may have a length L₁ that is greater than 0.5 meter, such as 1 meter or more, and the second portion 303 may have a length L₂ that is less than 0.5 meter, such as between about 0.3 and 0.4 meters. The second portion 303 may have a width dimension W₂ that is greater than a width dimension W₁ of the first portion 302. For example, the first portion 302 may have a width W₁ that is less than 0.3 meters (e.g., 0.15-0.25 meters) and the second portion 303 may have a width W₂ that is greater than 0.3 meters (e.g., 0.3-0.4 meters or more).

The detector area may be produced by arranging an array of detector modules 304 in a desired geometric shape or pattern. Each module 304 may include an array of individual detector elements (pixels), each including a scintillator (e.g., gadolinium oxysulfide (GOS)) coupled to a photodiode, and including an electronics assembly for outputting digital image data. The modules 304 may be abutted along any of their edges to form a detector area having any arbitrary size and shape. In the embodiment of FIGS. 3A-3C, the first portion 302 of the detector area may be formed by abutting a group of modules 304 along the length dimension, L₁, and the width dimension, W₁. For example, the first portion 302 may include two adjacent rows of detector modules 304, each row being 55 modules in length, for a total of 110 modules. The first portion 302 may be a large field-of-view (e.g., providing ˜50 cm diameter or greater reconstruction volume), multi-slice (e.g., 64 slice) diagnostic-quality true CT detector.

The second portion 303 of the detector area may be formed by abutting additional row(s) of modules 304 in the width direction along a section of the modules 304 forming the first portion 302. In the embodiment of FIGS. 3A-3C, the second portion 303 of the detector area may be formed by abutting three additional rows of modules 304 on either side of a central section of the two rows of modules 304 forming the first portion 302. The length of the central section may define the length dimension, L₂, of the second portion 303. The distance between the edges of the outer two rows of modules 304 in the second portion 303 may define the width dimension, W₂, of the second portion 303. In the embodiment of FIGS. 3A-3C, the second portion 303 of the detector area includes eight adjacent rows of detector modules 304, each row being 19 modules in length, for a total of 152 modules. The second portion 303 may be a rectangular panel detector that may be used for 2D x-ray fluoroscopy and/or cone-beam CT imaging.

The detector modules 304 in the detector system 301 may have a uniform size and shape or may have varying size(s) and/or shape(s). In one embodiment, the modules 304 may be a 2D element array, with for example 640 pixels per module (e.g., 32×20 pixels). The modules 304 may be mounted within a housing of a detector chassis 305, which may include a rigid frame comprised of a suitable structural material (e.g., aluminum) that supports the array of detector modules 304. In embodiments, the modules 304 may be enclosed within a light-tight housing (not illustrated in FIGS. 3A-3C for clarity). The modules 304 may be supported by the chassis 305 such that the modules 304 are curved or angled along the length of the chassis 305 to form or approximate a semicircular arc, with the arc center coinciding with a focal spot 307 of the x-ray source 43. In some embodiments, the modules 304 of the second portion 303 may additionally be curved or angled along the width of the chassis 305 to form or approximate a semicircular arc centered on the focal sport 307 of the x-ray source 43. The modules 304 of the second portion 303 may thus approximate a portion of a spherical surface that is centered on the focal spot 307 of the x-ray source 43. The detector system 301 may also include an anti-scatter assembly (not shown) located over the detector modules 304. The anti-scatter assembly may include a two-dimensional grid comprised of x-ray absorbent material located between the columns and rows of detector elements (pixels) or may be an array of x-ray absorbent plates located between adjacent columns or rows of detector elements.

In embodiments, the x-ray source 43 of the imaging system 100 may include an adjustable collimator 306 that defines the shape of an x-ray beam 317 emitted by the source 43. The collimator 306 may include motor-driven shutters or leaves comprised of an x-ray absorbent material (e.g., lead or tungsten) that may block a portion of the x-rays generated by an x-ray tube. In a first configuration shown in FIG. 3B, the collimator 306 may collimate the beam 317 so that it covers the first portion 302 of the detector area. The first configuration shown in FIG. 3B may be utilized, for example, for performing large field-of-view fan-beam helical or axial CT scans.

In a second configuration shown in FIG. 3C, the collimator 306 may collimate the beam 317 so that it covers the second portion 303 of the detector area. The second configuration shown in FIG. 3C may be utilized for performing 2D fluoroscopic imaging and/or 3D cone-beam CT scans. In embodiments, the configuration of the collimator 306 may be adjusted by a system controller, which may be implemented on a computer (e.g., computer 46). The system controller may also send a configuration signal to the detector system 301 to indicate the detector modules 304 from which to read out image data based on the shape of the x-ray beam 317. The imaging device 100 including an imaging gantry 40 as shown in FIGS. 3A-3C may be used to perform diagnostic-quality CT scans (e.g., multi-slice large field-of-view axial and/or helical scans), 2D fluoroscopic imaging and/or 3D cone beam CT imaging using a single x-ray source 43, high-voltage generator 44 and detector system 301.

It will be understood that other configurations of an imaging gantry 40 may be used in an imaging device 100 in accordance with various embodiments. For example, rather than a single detector system 301 that includes an elongated first portion 302 and a wider, panel-shaped second portion 303 as shown in FIGS. 3A-3C, the imaging gantry 40 may include separate detectors for performing fan-beam CT imaging and x-ray fluoroscopy. An example of an imaging gantry having multiple detectors, and optionally multiple x-ray sources, for performing different x-ray imaging modalities is disclosed in U.S. Pat. No. 9,526,461 B2, the entire contents of which are incorporated herein.

It will be further understood that as an alternative or addition to an imaging gantry 40 that includes at least one x-ray source 43 and at least one x-ray detector system 301, an imaging gantry 40 for an embodiment imaging device 100 may include other imaging components, such as magnetic resonance imaging (MM) components (e.g., magnet, gradient coil, RF coil), nuclear imaging components (e.g., gamma camera for PET and/or SPECT imaging), an ultrasound transducer, or optical imaging components (e.g., optical radiation source(s) and camera(s)). The imaging components on the imaging gantry 40 may be rotatable around the imaging gantry 40 such as on a rotor 41 as shown in FIGS. 3A-3C, or may be fixed on the imaging gantry 40.

FIGS. 4A-4D illustrate another embodiment of a medical imaging device 400 that may be similar to imaging device 100 shown in FIGS. 1A-1E. The imaging device 400 includes a generally O-shaped imaging gantry 40 defining a bore 61 having an isocenter 62 and including image collection components, as described above. The imaging gantry 40 is suspended from the support surface 151 by the support column 101, which may be attached to the linear motion system 103. As in the imaging device 100 of FIGS. 1A-1E, the linear motion system 103 of imaging device 400 may be a ceiling-mounted two-axis linear stage system (see FIG. 4D) that enables translation of the imaging gantry 40 in two perpendicular directions, and the support column 101 may include a second linear motion system, such as a telescoping portion of the support column 101, that enables the imaging gantry 40 to translate in a third perpendicular direction. In the imaging device 400 shown in FIGS. 4A-4D, the imaging gantry 40 is attached to the end of the support column 101, rather than to a gimbal 30 as in the embodiment of FIGS. 1A-1E.

The support column 101 may include a joint 401 that enables the imaging gantry 40 to rotate (i.e. pivot) in two perpendicular directions with respect to the base end 111 of the support column 101. The joint 401 may include a segment of the support column 101 that includes a pair of wedge-shaped outer members 403a, 403b between two base members 405a, 405b. A universal joint (not visible) located inside the wedge-shaped outer members 403a, 403b connects the base members 405a, 405b. The universal joint may include a pair of shafts each having a yoke connected by a cross-shaft that allows the shafts to pivot relative to one another in two mutually-perpendicular directions. The wedge-shaped outer members 403a, 403b are rotatable in the directions of arrows 406 and 408, respectively. The wedge-shaped outer members 403a, 403b may rotate independently of each another and each of the base members 405a, 405b. Drive motors 416, 418 in the support column 101 may drive the rotation of each of the wedge-shaped outer members 403a, 403b. The joint 401 further includes an angled interfacing surface 404 between the wedge-shaped outer members 405a, 405b. Relative rotation of the wedge-shaped outer members 403a, 403b changes the orientation of the distal base member 405b (i.e., nearest to the imaging gantry 40) relative to the proximal base member 405a (i.e., nearest to the base end 111 of the support column 101). As the wedge-shaped outer members 403a, 403b rotate, the shafts of the universal joint pivot in response to the change in relative orientation of the base members 405a, 405b while simultaneously preventing the base members 405a, 405b from moving torsionally (i.e., twisting) relative to one another. The wedge-shaped outer members 403a, 403b may rotate continuously to pivot the imaging gantry 40 along two perpendicular directions without causing any cables extending through the support column 101 and joint 401 becoming twisted.

By controlling the relative rotation of the wedge-shaped outer members 403a, 403b, the distal end 113 of the support column 101 and imaging gantry 40 may rotate in two perpendicular directions relative to base end 111 of the support column 101 as illustrated by arrows 407 and 409 in FIG. 4A. For example, from an initial configuration as shown in FIG. 4A, rotating the wedge-shaped outer members 403a, 403b at the same velocity in opposite directions causes the imaging gantry 40 to pivot back and forth with respect the base end 111 of the support column 101 along the direction of arrow 407. When the wedge-shaped outer members 403a, 403b are rotated in opposite directions ±90° from the initial configuration, the imaging gantry 40 is at its maximum pivot angle with respect to the base end 111 of the support column 101. The magnitude of the maximum pivot angle of the imaging gantry 40 is defined by the angle of the interfacing surface 404 of the wedge-shaped outer members 403a, 403b. In embodiments, the imaging gantry 40 may be pivotable at least ±15°, such as ±30°, ±45°, ±60°, ±90° or more, with respect to the base end 111 of the support column 101.

When the wedge-shaped outer members 403a, 403b are rotated at the same velocity in the same direction, the magnitude of the pivot angle of the imaging gantry 40 with respect to the base end 111 of the support column 101 remains constant while the direction in which the gantry pivots is rotated 0-360° around the base end 111 of the support column 101. In the embodiment of FIG. 4A, for example, when the wedge-shaped outer members 403a, 403b are first rotated in the same direction ±90° from the initial configuration, and are then rotated in opposite directions at the same velocity, the imaging gantry 40 will pivot back and forth along the direction of arrow 409.

The pivoting of the imaging gantry 40 along the direction of arrows 409 and/or 407 may also be coordinated with translational movement of the imaging gantry 40 along direction of arrows 105, 107 and/or 109 (see FIG. 1A) so that the rotation of the imaging gantry 40 is about the center of the bore 61 of the imaging gantry 40. In the embodiment of FIG. 4A, for example, pivoting the imaging gantry 40 about the joint 401 along the direction of arrow 406 would cause the isocenter of the imaging gantry 40 to shift slightly, both along the length of the patient 200 and in the vertical direction. To compensate for this shift, the motion controller 203 may control the translation drive motors 108, 110 and/or 112 to translate the imaging gantry 40 by the same magnitude in which the isocenter would shift, but in the opposite direction, so that the isocenter remains in the same location with respect to the patient 200 while the imaging gantry 40 pivots along the direction of arrow(s) 407 and/or 409.

The imaging device 400 may further include a rotary bearing 411 that may enable the imaging gantry 40 to rotate with respect to base member 405b in the direction of arrow 412. The rotary bearing 411 may enable the imaging gantry 40 to rotate with respect to base member 405b of joint 401 provide the imaging gantry 40 with a third rotational degree-of-freedom. The axis of rotation of the rotary bearing 411 may be aligned with the center of the bore 61 of the imaging gantry 40, so that the rotation of the imaging gantry 40 along the direction of arrow 412 is isocentric. A rotation drive motor 413 may drive the rotation of the imaging gantry 40 on rotary bearing 411 along the direction of arrow 412. A motion controller 203 (shown schematically) may be operatively coupled to each of the drive motors 108, 110, 112, 416, 418, and 413 of the imaging device 400, and may control various translational and rotational movements of the imaging gantry 40 as described above. The operation of the imaging device 400 may be similar to the operation of the imaging device 100 described with reference to FIGS. 1A-1E and 3A-3C, and the imaging device 400 in some embodiments may include one or more of at least one force sensor, at least one proximity sensor and a 3D motion control input device 209 such as shown in FIGS. 2A-2B.

FIGS. 4A-4D illustrate the imaging gantry 40 of imaging device 400 moved to different positions relative to a patient 200 supported on a patient positioner 201. As shown in FIG. 4A, the imaging device 400 may be used to obtain images from a patient 200 in a lying position, such as a CT scan along the length of the patient 200 on a horizontal patient table. The imaging gantry 40 may also be rotated relative to the support column 101 to obtain images from a patient 200 in a standing position as shown in FIG. 4B, or in an inclined position as shown in FIGS. 4C and 4D. The imaging gantry 40 may be rotated so that the bore 61 is offset from the support column 101 as shown in FIGS. 4B-4D which may facilitate imaging a standing or inclined patient without interference from the support column 101. The absence of a gimbal 30 in the imaging device 400 may also reduce obstructions proximate to the bore 61. The imaging gantry 40 may be oriented perpendicular to the patient 200 as shown in FIGS. 4A, 4C and 4D or at an oblique angle relative to the patient as shown in FIG. 4B.

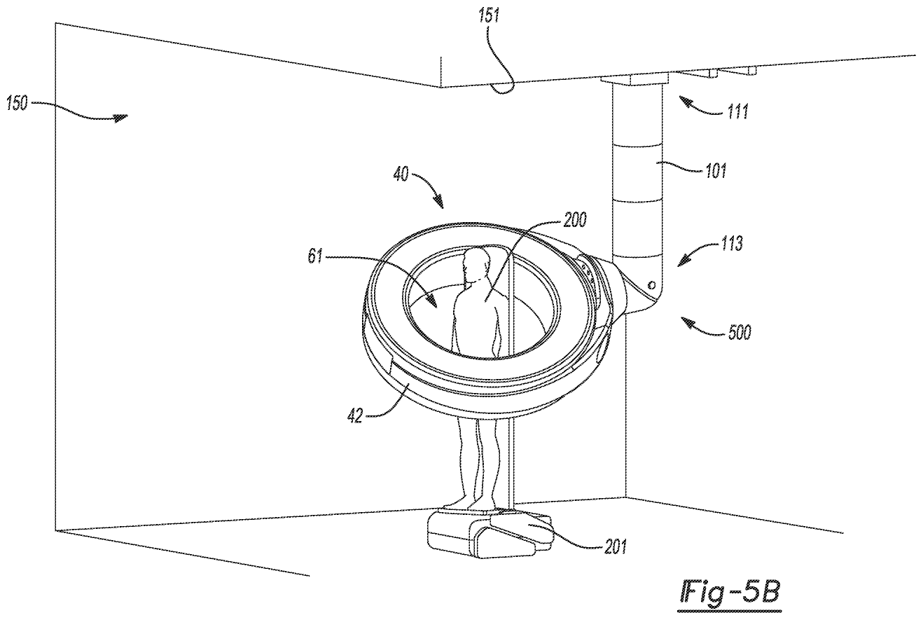

FIGS. 5A and 5B illustrate yet another embodiment of a medical imaging device 500. As with the embodiments shown in FIGS. 1A-1E and 4A-4D, the imaging device 500 includes a generally O-shaped imaging gantry 40 defining a bore 61 and including image collection components, as described above. The imaging gantry 40 is attached to the distal end 113 of a support column 101 that may suspend the imaging gantry 40 from a support surface 151 (e.g., a ceiling). As with the embodiments shown in FIGS. 1A-1E and 4A-4D, the imaging gantry 40 may be translated in three perpendicular directions with respect to the support surface 151. A two-axis linear stage system may enable the imaging gantry 40 and support column 101 to move in two perpendicular directions, and a telescoping portion of the support column 101 may enable the imaging gantry 40 to move in a third direction.

In the embodiment of the imaging device 500 shown in FIGS. 5A-5B, the rotational degrees of freedom of the imaging gantry 40 relative to the support surface 151 may be provided by three rotary bearings 501, 503 and 505. A first rotary bearing 501 may be located in the support column 101 as shown in FIG. 5A. Alternately, the first rotary bearing 501 may be located between the base end 111 of the support column 101 and the support surface 151. A first rotation drive motor 502 may drive the rotation of the distal end 113 of the support column 101 and the imaging gantry 40 with respect to the support surface 151 along the direction of arrow 504.

A second rotary bearing 503 may be located in the support column 101 between the first rotary bearing 501 and the distal end 113 of the support column 101. A second rotation drive motor 506 may drive the rotation of the imaging gantry 40 and the distal end 113 of the support column 101 with respect to the first rotary bearing 501 along the direction of arrow 508. The axes of rotation of the first and second rotary bearings 501, 503 may be perpendicular to one another. As shown in FIG. 5A, the axis of rotation of rotary bearing 501 is along the length of the support column 101 while the axis of rotation of rotary bearing 503 is perpendicular to the length of the support column 101.

The imaging device 500 includes a third rotary bearing 505 located between the second rotary bearing 503 and the imaging gantry 40. As shown in FIG. 5A, the third rotary bearing 505 is located at the interface between the distal end 113 of the support column 101 and the imaging gantry 40. Alternately, the third rotary bearing 505 may be located in the support column 101. A third rotation drive motor 510 may drive the rotation of the imaging gantry 40 on the third rotary bearing 505 in the direction of arrow 512. The axis of rotation of the third rotary bearing 505 may be perpendicular to the axis of rotation of the second rotary bearing 503. The axis of rotation of the third rotary bearing 505 may also be aligned with the center of the bore 61 of the imaging gantry 40 so that the rotation of the imaging gantry 40 along the direction of arrow 512 is isocentric.

The combination of rotary bearings 501, 503 and 505 enable the imaging gantry 40 to rotate about three axes. For example, rotation about the first rotary bearing 501 causes the imaging gantry 40 to rotate about the vertical axis. Rotation about the second rotary bearing 503 causes the imaging gantry 40 to pivot about a first horizontal axis (i.e., into and out of the page in the configuration shown in FIG. 5A). To pivot the imaging gantry about the second horizontal axis (i.e., from left to right in the configuration shown in FIG. 5A), the second rotary bearing 503 may be rotated with respect to the first 501 and third 505 rotary bearings so that the gantry will pivot on the second rotary bearing 503 in the transverse direction (i.e., to the left and right of the page in FIGS. 5A-5B). Further, as in the case of the embodiment imaging device 400 shown in FIGS. 4A-4D, the controller of imaging device 500 may coordinate the rotational and translational movements of the imaging gantry 40 to control the imaging gantry 40 to rotate about its isocenter in all three rotational degrees-of-freedom.

A controller may be operatively coupled to each of the drive motors 502, 506, 510 of the imaging device 500, and may control various translational and rotational movements of the imaging gantry 40 as described above. The operation of the imaging device 500 may be similar to the operation of the imaging device 100 described with reference to FIGS. 1A-1E, 3A-3C and 4A-4D, and the imaging device 500 in some embodiments may include one or more of at least one force sensor, at least one proximity sensor and a 3D motion control input device 209 such as shown in FIGS. 2A-2B.

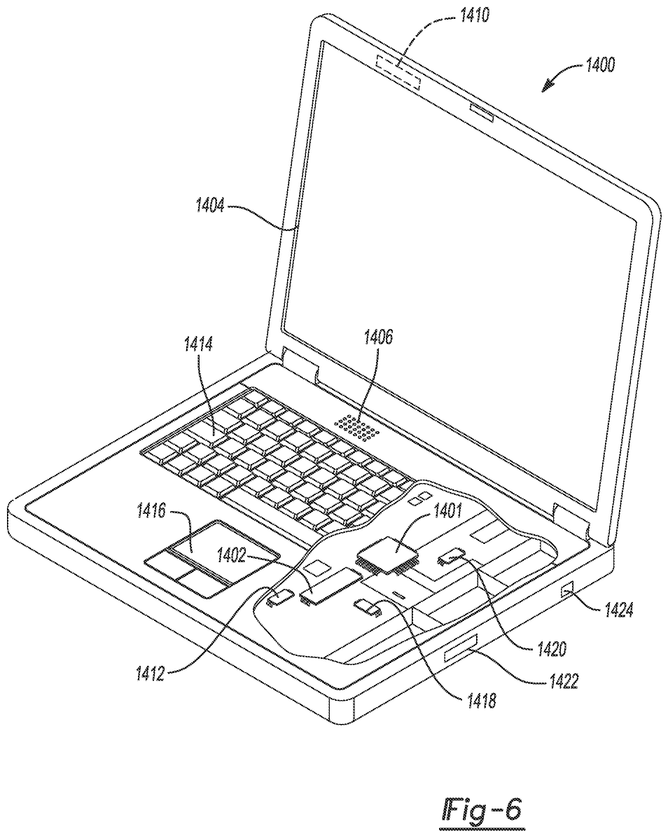

FIG. 6 is a schematic diagram of a computing device 1400 useful for performing and implementing the various embodiments described above. The computing device 1400 may perform the functions of a controller for an imaging device as illustrated and described herein. While the computing device 1400 is illustrated as a laptop computer, a computing device providing the functional capabilities of the computer device 1400 may be implemented as a workstation computer, an embedded computer, a desktop computer, a server computer, or a handheld computer (e.g., tablet, smartphone, etc.). A typical computing device 1400 may include a processor 1401 coupled to an electronic display 1404, a speaker 1406, and a memory 1402, which may be a volatile memory as well as a nonvolatile memory (e.g., a disk drive). When implemented as a laptop computer or desktop computer, the computing device 1400 may also include a removable media drive 1422 such as a CD/DVD drive, an SD card reader, and other removable flash drives coupled to the processor 1401. The computing device 1400 may include an antenna 1410, a multimedia receiver 1412, a transceiver 1418 and/or communications circuitry coupled to the processor 1401 for sending and receiving electromagnetic radiation, connecting to a wireless data link, and receiving data. Additionally, the computing device 1400 may include network access ports 1424 coupled to the processor 1401 for establishing data connections with a network (e.g., LAN coupled to a service provider network, etc.). When implemented as a laptop computer or a desktop computer the computing device 1400 typically also includes a keyboard 1414 and a touch pad 1416 for receiving user inputs.

The foregoing method descriptions are provided merely as illustrative examples and are not intended to require or imply that the steps of the various embodiments must be performed in the order presented. As will be appreciated by one of skill in the art the order of steps in the foregoing embodiments may be performed in any order. Words such as “thereafter,” “then,” “next,” etc. are not necessarily intended to limit the order of the steps; these words may be used to guide the reader through the description of the methods. Further, any reference to claim elements in the singular, for example, using the articles “a,” “an” or “the” is not to be construed as limiting the element to the singular.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The hardware used to implement the various illustrative logics, logical blocks, modules, and circuits described in connection with the aspects disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Alternatively, some steps or methods may be performed by circuitry that is specific to a given function.

In one or more exemplary aspects, the functions described may be implemented in hardware, software, firmware, or any combination thereof. If implemented in software, the functions may be stored on as one or more instructions or code on a non-transitory computer-readable medium. The steps of a method or algorithm disclosed herein may be embodied in a processor-executable software module executed which may reside on a non-transitory computer-readable medium. Non-transitory computer-readable media includes computer storage media that facilitates transfer of a computer program from one place to another. A storage media may be any available media that may be accessed by a computer. By way of example, and not limitation, such non-transitory computer-readable storage media may comprise RAM, ROM, EEPROM, CD-ROM or other optical disk storage, magnetic disk storage or other magnetic storage devices, or any other medium that may be used to carry or store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of non-transitory computer-readable storage media. Additionally, the operations of a method or algorithm may reside as one or any combination or set of codes and/or instructions on a machine readable medium and/or computer-readable medium, which may be incorporated into a computer program product.

The preceding description of the disclosed aspects is provided to enable any person skilled in the art to make or use the present disclosure. Various modifications to these aspects will be readily apparent to those skilled in the art, and the generic principles defined herein may be applied to other aspects without departing from the scope of the disclosure. Thus, the present disclosure is not intended to be limited to the aspects shown herein but is to be accorded the widest scope consistent with the principles and novel features disclosed herein.

It will be further appreciated that the terms “include,” “includes,” and “including” have the same meaning as the terms “comprise,” “comprises,” and “comprising.” Moreover, it will be appreciated that terms such as “first,” “second,” “third,” and the like are used herein to differentiate certain structural features and components for the non-limiting, illustrative purposes of clarity and consistency.

The present disclosure also comprises the following clauses, with specific features laid out in dependent clauses, that may specifically be implemented as described in greater detail with reference to the configurations and drawings above.

### CLAUSES

I. A medical imaging device, comprising:
an imaging gantry attached to a support surface, the imaging gantry comprising an O-shaped housing defining a bore and containing one or more image collection components configured to obtain imaging data from a patient located in the bore;
a support column that supports the imaging gantry relative to the support surface; and
a drive system comprising at least one drive motor that is operable to translate the imaging gantry along three perpendicular directions relative to the support surface and rotate the imaging gantry about three perpendicular axes relative to the support surface.
II. The medical imaging device of any of the preceding clauses, further comprising a controller coupled to the drive system and configured to send control signals to the at least one drive motor of the drive system to control the translation and rotation of the imaging gantry relative to the support surface.
III. The medical imaging device of any of the preceding clauses, wherein the controller is configured to send control signals to the at least one drive motor of the drive system to rotate the imaging gantry about three perpendicular axes relative to the center of the bore of the imaging gantry.
IV. The medical imaging device of any of the preceding clauses, wherein the imaging gantry is suspended from the support surface by the support column.
V. The medical imaging device of any of the preceding clauses, wherein the support surface comprises the ceiling of a hybrid operating room.
VI. The medical imaging device of any of the preceding clauses, wherein the image collection components comprise at least one of: (i) an x-ray source and x-ray detector array, (ii) a gamma-ray camera, and (iii) magnetic resonance imaging components.
VII. The medical imaging device of any of the preceding clauses, wherein the image collection components comprise at least one x-ray source and at least one x-ray detector array, and the medical imaging device is configured to obtain imaging data including fan-beam computed tomography (CT) scan data and x-ray fluoroscopic image data.
VIII. The medical imaging device of any of the preceding clauses, wherein the image collection components comprise:
an x-ray source; and
an x-ray detector array that includes a first portion having a first length and a first width and a second portion having a second length and a second width, and the first length is greater than the second length and the second width is greater than the first width.
IX. The medical imaging device of any of the preceding clauses, wherein the x-ray source comprises an adjustable collimator that is configured to shape a beam of x-ray radiation from the x-ray source to project onto the first portion of the detector array during a fan-beam CT imaging scan and to shape a beam of x-ray radiation from the x-ray source to project onto the second portion of the detector array during x-ray fluoroscopic imaging and/or a cone-beam CT imaging scan.
X. The medical imaging device of any of the preceding clauses, further comprising a first linear motion system mounted to the support surface that enables the imaging gantry and the support column to translate along two perpendicular directions relative to the support surface.
XI. The medical imaging device of any of the preceding clauses, wherein the first linear motion system comprises a two-axis linear stage system.
XII. The medical imaging device of any of the preceding clauses, wherein the drive system comprises a first translation drive motor that drives the translation of the imaging gantry and the support column on the first linear motion system along a first direction, and a second translation drive motor that drives the translation of the imaging gantry and the support column on the first linear motion system along a second direction that is perpendicular to the first direction.
XIII The medical imaging device of any of the preceding clauses, further comprising a second linear motion system on the support column that enables the imaging gantry to translate along a third perpendicular direction relative to the support surface.
XIV. The medical imaging device of any of the preceding clauses, wherein the second linear motion system comprises a telescoping portion of the support column.
XV. The medical imaging device of any of the preceding clauses, wherein the drive system comprises a third translation drive motor that drives the translation of the imaging gantry on the second linear motion system along a third direction perpendicular to the first direction and the second direction.
XVI. The medical imaging device of any of the preceding clauses, wherein the controller is configured to receive feedback indicating the position of a patient positioner that supports the patient and to control motion of the imaging gantry based on the position and/or motion of the patient positioner.
XVII. The medical imaging device of any of the preceding clauses, wherein the controller is configured to implement a collision model to control motion of the imaging gantry to prevent the imaging gantry from colliding with the patient positioner or the patient.
XVIII. The medical imaging device of any of the preceding clauses, wherein the controller is coupled to one or more proximity sensors that prevent the imaging gantry from colliding with another object.
XIX. The medical imaging device of any of the preceding clauses, wherein the controller is coupled to one or more force sensors that measure forces or torques applied to or by the medical imaging device.
XX. The medical imaging device of any of the preceding clauses, wherein the controller is configured to receive feedback from the one or more force sensors indicating a force and/or torque applied to the imaging gantry, and in response to the feedback, control the drive system to translate and/or rotate the imaging gantry in the direction of the applied force and/or torque.
XXI. The medical imaging device of any of the preceding clauses, further comprising:
a three-dimensional motion control input device coupled to a controller, the three-dimensional motion control input device comprising:
   a base;
   a moveable element on the base, where the moveable element is configured to be manipulated by a user to translate along three perpendicular directions relative to the base and is rotate about three perpendicular axes relative to the base; and
   an electronic circuit that generates control signals in response to translation and/or rotational movement of the moveable element with respect to the base,
wherein the controller is configured to receive the control signals generated by the three-dimensional motion control input device and control the drive system to translate and/or rotate the imaging gantry based on the translation and/or rotation of the moveable element relative to the base.
XXII. The medical imaging device of any of the preceding clauses, wherein the three-dimensional motion control input device is located on the medical imaging device.
XXIII The medical imaging device of any of the preceding clauses, further comprising a gimbal attached to a distal end of the support column and including a pair of arms extending away from the support column, each arm of the gimbal connected to an opposite side of the imaging gantry by a pair of rotary bearings that enable the imaging gantry to rotate with respect to the gimbal about a first axis, wherein the drive system comprises a first rotation drive motor that drives the rotation of the imaging gantry about the first axis.
XXIV. The medical imaging device of any of the preceding clauses, wherein the gimbal and the imaging gantry are attached to the support surface via a rotary bearing that enables the gimbal and the imaging gantry to rotate with respect to the support surface about a second axis that is perpendicular to the first axis, wherein the drive system comprises a second rotation drive motor that drives the rotation of the gimbal and the imaging gantry about the second axis.
XXV. The medical imaging device of any of the preceding clauses, wherein the rotary bearing that enables the gimbal and the imaging gantry to rotate about the second axis is located at the interface between the gimbal and the distal end of the support column, within the support column, or at the interface between a base end of the support column and the first linear motion system.
XXVI. The medical imaging device of any of the preceding clauses, further comprising a curved bearing assembly between the distal end of the support column and the gimbal that enables the gimbal and the imaging gantry to rotate with respect to the support surface about a third axis that is perpendicular to the first axis and the second axis, wherein the drive system comprises a third rotation drive motor that drives the rotation of the gimbal and the imaging gantry about the second axis.
XXVII. The medical imaging device of any of the preceding clauses, wherein the first axis, the second axis, and the third axis intersect at the center of the bore of the imaging gantry.
XXVIII. The medical imaging device of any of the preceding clauses, wherein the imaging gantry is attached to a distal end of the support column, and the support column comprises a joint that enables the imaging gantry to rotate about a first axis and a second axis relative to the support surface, where the first axis is perpendicular to the second axis.
XXIX. The medical imaging device of any of the preceding clauses, wherein the joint is a segment of the support column, and the joint comprises:
first and second wedge-shaped outer members having angled interfacing surfaces, the first and second wedge-shaped outer members located between first and second base members; and
a universal joint located interior of the first and second wedge-shaped outer members and connecting the first and second base members so as to inhibit torsional motion between the respective first and second base members, wherein the drive system comprises first and second rotation drive motors coupled to the respective first and second wedge-shaped outer members, the first rotation drive motor driving the rotation of the first wedge-shaped outer member relative to the first and second base members and the second wedge-shaped outer member, and the second rotation drive motor driving the rotation of the second wedge-shaped outer member relative to the first and second base members and the first wedge-shaped outer member, to cause the first and second base members to pivot relative to each other about the first axis and the second axis.
XXX. The medical imaging device of any of the preceding clauses, wherein the imaging gantry is attached to the support surface via a rotary bearing that enables the imaging gantry to rotate with respect to the support surface about a third axis, wherein the drive system comprises a third rotation drive motor that drives the rotation of the imaging gantry about the third axis.
XXXI. The medical imaging device of any of the preceding clauses, wherein the rotary bearing is located at the interface between the distal end of the support column and the imaging gantry.
XXXII. The medical imaging device of any of the preceding clauses, wherein the third axis extends through the center of the bore of the imaging gantry.
XXXIII. The medical imaging device of any of the preceding clauses, wherein the controller is configured to send control signals to the drive system to coordinate rotational and translational movements of the imaging gantry such that as the imaging gantry rotates about the first axis and/or the second axis, the center of the bore of the imaging gantry remains stationary relative to the support surface.
XXXIV. The medical imaging device of any of the preceding clauses, wherein the imaging gantry is attached to a distal end of the support column, and the medical imaging device further comprises:
a first rotary bearing located between the support surface and the distal end of the support column, the first rotary bearing enabling the imaging gantry to rotate with respect to the support surface about a first axis;
a second rotary bearing located between the first rotary bearing and the distal end of the support column, the second rotary bearing enabling the imaging gantry to rotate with respect to the support surface about a second axis that is perpendicular to the first axis; and
a third rotary bearing located between the second rotary bearing and the imaging gantry, the third rotary bearing enabling the imaging gantry to rotate with respect to the support surface about a third axis that is perpendicular to the second axis,
wherein the drive system comprises a first rotation drive motor that drives the rotation of the imaging gantry about the first axis, a second rotation drive motor that drives the rotation of the imaging gantry about the second axis and a third rotation drive motor that drives the rotation of the imaging gantry about the third axis.
XXXV. The medical imaging device of any of the preceding clauses, wherein the first axis extends along a length of the support column, the second axis extends transverse to the length of the support column and the third axis extends through the center of the bore of the imaging gantry.
XXXVI. The medical imaging device of any of the preceding clauses, wherein the controller is configured to send control signals to the drive system to coordinate rotational and translational movements of the imaging gantry such that as the imaging gantry rotates about the first axis and/or the second axis, the center of the bore of the imaging gantry remains stationary relative to the support surface.
XXXVII. A medical imaging device usable in a medical facility having a support surface, the medical imagining device comprising:
an imaging gantry supported by the support surface, the imaging gantry comprising a gantry housing defining a bore and supporting one or more image collection components configured to obtain image data of a patient positioned in the bore;
a support column coupled between the imaging gantry and the support surface; and
a drive system operably coupled to the imaging gantry and configured to effect translation of the imaging gantry relative to the support surface, the drive system comprising;
   a translation drive motor; and
   a control system comprising one or more controllers, the control system comprising a motion controller in communication with the translation drive motor and configured to send control signals to the translation drive motor to control movement of the imaging gantry relative to the support surface.
XXXVIII. The medical imaging device of any of the preceding clauses, wherein the drive system is further configured to effect rotation of the imaging gantry relative to the support surface and further comprises a rotation drive motor, wherein the motion controller is configured to send control signals to the translation drive motor and the rotation drive motor to control movement of the imaging gantry relative to the support surface.
XXXIX. The medical imaging device of any of the preceding clauses, wherein the imaging gantry defines a pitch axis, a roll axis, and a yaw axis that intersect perpendicular to each other at a center of the bore, and wherein the rotation drive motor is further defined as a pitch rotation drive motor, and yaw rotation drive motor, and a roll rotation drive motor.
XL. The medical imaging device of any of the preceding clauses, further comprising a gimbal coupled to the support column and rotatably coupled to the imaging gantry such that the imaging gantry is rotatable with respect to the support column about the pitch axis, and wherein the pitch rotation drive motor rotates the imaging gantry about the pitch axis.
XLI. The medical imaging device of any of the preceding clauses, further comprising a rotary bearing assembly coupled to the support column that enables the imaging gantry to rotate with respect to the support surface about the yaw axis, and wherein the yaw rotation drive motor rotates the imaging gantry about the yaw axis.
XLII. The medical imaging device of any of the preceding clauses, further comprising a curved bearing assembly coupled between the support column and the imaging gantry that enables the imaging gantry to rotate with respect to the support surface about the roll axis, and wherein the roll rotation drive motor rotates the imaging gantry about the roll axis.
XLIII. The medical imaging device of any of the preceding clauses, further comprising a first linear motion system coupled between the support surface and the support column and configured to constrain movement of the imaging gantry and the support column relative to the support surface in two degrees of freedom.
XLIV. The medical imaging device of any of the preceding clauses, further comprising a second linear motion system coupled between the support surface and the imaging gantry and configured to constrain movement of the imaging gantry relative to the support surface in a third degree of freedom.
XLV. The medical imaging device of any of the preceding clauses, wherein the controller is further configured to send control signals to the drive system to coordinate rotational and translational movements of the imaging gantry such that as the imaging gantry moves about a first axis and a second axis, a center of the bore of the imaging gantry remains stationary relative to the support surface.
XLVI. The medical imaging device of any of the preceding clauses, further comprising a patient positioner configured to support the patient, wherein the patient positioner is movable between at least a first patient position and a second patient position to move the patient relative to the support surface.
XLVII. The medical imaging device of any of the preceding clauses, wherein the control system is configured to receive feedback comprising position data of the patient positioner and to control movement of the imaging gantry based on the position of the patient positioner.
XLVIII. The medical imaging device of any of the preceding clauses, wherein the position data of the patient positioner comprises movement data, and wherein the control system is further configured to control movement of the imaging gantry based on the position and movement of the patient positioner to effect coordinated motion.
XLIX. The medical imaging device of any of the preceding clauses, wherein the image collection components comprise at least one of an x-ray source and x-ray detector array.
L. The medical imaging device of any of the preceding clauses, wherein the medical imaging device is configured to obtain image data including fan-beam computed tomography (CT) scan data and x-ray fluoroscopic image data.
LI. The medical imaging device of any of the preceding clauses, wherein the x-ray detector array includes a first portion having a first length and a first width and a second portion having a second length and a second width, wherein the first length is greater than the second length and the second width is greater than the first width.
LII. The medical imaging device of any of the preceding clauses, wherein the x-ray source comprises an adjustable collimator that is configured to shape a beam of x-ray radiation from the x-ray source to project onto the first portion of the detector array during a fan-beam CT imaging scan and to shape a beam of x-ray radiation from the x-ray source to project onto the second portion of the detector array during x-ray fluoroscopic imaging and/or a cone-beam CT imaging scan.
LIII. The medical imaging device of any of the preceding clauses, further comprising a robotic arm coupled to the imaging gantry for positioning an end effector usable during a medical procedure, wherein the robotic arm is movable between at least a first arm pose and a second arm pose.
LIV. The medical imaging device of any of the preceding clauses, wherein the control system is configured to receive feedback comprising pose data of the robotic arm and to control movement of the imaging gantry based on the position of the robotic arm.
LV. The medical imaging device of any of the preceding clauses, wherein the pose data of the robotic arm comprises movement data, and wherein the control system is further configured to control movement of the imaging gantry based on the pose and movement of the robotic arm to effect coordinated motion.
LVI. A medical imaging device usable in a medical facility having a support surface, the medical imagining device comprising:
an imaging gantry supported by the support surface, the imaging gantry comprising a gantry housing defining a bore and supporting one or more image collection components configured to obtain image data of a patient positioned in the bore;
a robotic arm coupled to the imaging gantry for positioning an end effector usable during a medical procedure;
a drive system operably coupled to the imaging gantry and comprising a drive motor configured to effect movement of the imaging gantry relative to the support surface; and
a control system comprising one or more controllers in communication with the drive motor and the robotic arm, the control system configured to send control signals to the translation drive motor to control movement of the imaging gantry relative to the support surface.
LVII. The medical imaging device of any of the preceding clauses, wherein the control system is further configured to send control signals to the robotic arm to simultaneously control relative movement of the imaging gantry and the robotic arm.
LVIII. A medical imaging device usable in a medical facility having a support surface, the medical imagining device comprising:
an imaging gantry supported by the support surface, the imaging gantry comprising a gantry housing defining a bore and supporting one or more image collection components configured to obtain image data of a patient positioned in the bore;
a drive system operably coupled to the imaging gantry and comprising a drive motor configured to effect movement of the imaging gantry relative to the support surface;
a patient positioner movable between a first patient support position and a second patient support position to move the patient relative to the support surface; and
a control system including one or more controllers in communication with the drive motor and the patient positioner, the control system configured to send control signals to the patient positioner and the translation drive motor to control movement of the imaging gantry and the patient positioner relative to the support surface.

What is claimed is:

1. A medical imaging device usable in a medical facility having a support surface, the medical imagining device comprising:
an imaging gantry supported by the support surface, the imaging gantry comprising a gantry housing defining a bore and supporting one or more image collection components configured to obtain image data of a patient positioned in the bore;
a support column coupled between the imaging gantry and the support surface; and
a drive system operably coupled to the imaging gantry and configured to effect translation of the imaging gantry relative to the support surface, the drive system comprising;
a translation drive motor; and
a control system comprising one or more controllers, the control system comprising a motion controller in communication with the translation drive motor and configured to send control signals to the translation drive motor to control movement of the imaging gantry relative to the support surface.

2. The medical imaging device of claim 1, wherein the drive system is further configured to effect rotation of the imaging gantry relative to the support surface and further comprises a rotation drive motor, wherein the motion controller is configured to send control signals to the translation drive motor and the rotation drive motor to control movement of the imaging gantry relative to the support surface.

3. The medical imaging device of claim 2, wherein the imaging gantry defines a pitch axis, a roll axis, and a yaw axis that intersect perpendicular to each other at a center of the bore, and wherein the rotation drive motor is further defined as a pitch rotation drive motor, and yaw rotation drive motor, and a roll rotation drive motor.

4. The medical imaging device of claim 3, further comprising a gimbal coupled to the support column and rotatably coupled to the imaging gantry such that the imaging gantry is rotatable with respect to the support column about the pitch axis, and wherein the pitch rotation drive motor rotates the imaging gantry about the pitch axis.

5. The medical imaging device of claim 3, further comprising a rotary bearing assembly coupled to the support column that enables the imaging gantry to rotate with respect to the support surface about the yaw axis, and wherein the yaw rotation drive motor rotates the imaging gantry about the yaw axis.

6. The medical imaging device of claim 3, further comprising a curved bearing assembly coupled between the support column and the imaging gantry that enables the imaging gantry to rotate with respect to the support surface about the roll axis, and wherein the roll rotation drive motor rotates the imaging gantry about the roll axis.

7. The medical imaging device of claim 2, further comprising a first linear motion system coupled between the support surface and the support column and configured to constrain movement of the imaging gantry and the support column relative to the support surface in two degrees of freedom.

8. The medical imaging device of claim 7, further comprising a second linear motion system coupled between the support surface and the imaging gantry and configured to constrain movement of the imaging gantry relative to the support surface in a third degree of freedom.

9. The medical imaging device of claim 2, wherein the motion controller is further configured to send control signals to the drive system to coordinate rotational and translational movements of the imaging gantry such that as the imaging gantry moves about a first axis and a second axis, a center of the bore of the imaging gantry remains stationary relative to the support surface.

10. The medical imaging device of claim 2, further comprising a patient positioner configured to support the patient, wherein the patient positioner is movable between at least a first patient position and a second patient position to move the patient relative to the support surface.

11. The medical imaging device of claim 10, wherein the control system is configured to receive feedback comprising position data of the patient positioner and to control movement of the imaging gantry based on the position of the patient positioner.

12. The medical imaging device of claim 11, wherein the position data of the patient positioner comprises movement data, and wherein the control system is further configured to control movement of the imaging gantry based on the position and movement of the patient positioner to effect coordinated motion.

13. The medical imaging device of claim 1, wherein the image collection components comprise at least one of an x-ray source and x-ray detector array.

14. The medical imaging device of claim 13, wherein the medical imaging device is configured to obtain image data including fan-beam computed tomography (CT) scan data and x-ray fluoroscopic image data.

15. The medical imaging device of claim 13, wherein the x-ray detector array includes a first portion having a first length and a first width and a second portion having a second length and a second width, wherein the first length is greater than the second length and the second width is greater than the first width.

16. The medical imaging device of claim 15, wherein the x-ray source comprises an adjustable collimator that is configured to shape a beam of x-ray radiation from the x-ray source to project onto the first portion of the detector array during a fan-beam CT imaging scan and to shape a beam of x-ray radiation from the x-ray source to project onto the second portion of the detector array during x-ray fluoroscopic imaging and/or a cone-beam CT imaging scan.

17. The medical imaging device of claim 2, further comprising a robotic arm coupled to the imaging gantry for positioning an end effector usable during a medical procedure, wherein the robotic arm is movable between at least a first arm pose and a second arm pose.

18. The medical imaging device of claim 17, wherein the control system is configured to receive feedback comprising pose data of the robotic arm and to control movement of the imaging gantry based on the position of the robotic arm.

19. The medical imaging device of claim 18, wherein the pose data of the robotic arm comprises movement data, and wherein the control system is further configured to control movement of the imaging gantry based on the pose and movement of the robotic arm to effect coordinated motion.

20. A medical imaging device usable in a medical facility having a support surface, the medical imagining device comprising:
an imaging gantry supported by the support surface, the imaging gantry comprising a gantry housing defining a bore and supporting one or more image collection components configured to obtain image data of a patient positioned in the bore;
a robotic arm coupled to the imaging gantry for positioning an end effector usable during a medical procedure;
a drive system operably coupled to the imaging gantry and comprising a drive motor configured to effect movement of the imaging gantry relative to the support surface; and
a control system comprising one or more controllers in communication with the drive motor and the robotic arm, the control system configured to send control signals to the drive motor to control movement of the imaging gantry relative to the support surface.

21. The medical imaging device of claim 20, wherein the control system is further configured to send control signals to the robotic arm to simultaneously control relative movement of the imaging gantry and the robotic arm.

22. A medical imaging device usable in a medical facility having a support surface, the medical imagining device comprising:
an imaging gantry supported by the support surface, the imaging gantry comprising a gantry housing defining a bore and supporting one or more image collection components configured to obtain image data of a patient positioned in the bore;
a drive system operably coupled to the imaging gantry and comprising a drive motor configured to effect movement of the imaging gantry relative to the support surface;
a patient positioner movable between a first patient support position and a second patient support position to move the patient relative to the support surface; and
a control system including one or more controllers in communication with the drive motor and the patient positioner, the control system configured to send control signals to the patient positioner and the drive motor to control movement of the imaging gantry and the patient positioner relative to the support surface.
